(19) United States
(12) Patent Application Publication
(10) Pub. No.: US 2022/0174979 A1
(43) Pub. Date: Jun. 9, 2022

(54) FORTIFIED HIGH PROTEIN FOOD & PRODUCTION METHOD

(71) Applicant: Erie Group International, Inc., Erie, IL (US)

(72) Inventors: James M. Klein, Geneseo, IL (US); Craig Air, Geneseo, IL (US)

(21) Appl. No.: 17/677,798

(22) Filed: Feb. 22, 2022

Related U.S. Application Data
(63) Continuation-in-part of application No. 15/650,737, filed on Jul. 14, 2017.
Application No. 15/650,737 is a continuation of application No. 14/875,463, filed on Oct. 5, 2015, now Pat. No. 9,723,859.
(60) Provisional application No. 63/200,184, filed on Feb. 19, 2021.
(60) Provisional application No. 62/059,355, filed on Oct. 3, 2014.

(51) Int. Cl. A23J 3/10; A23J 1/22; A23J 3/26

(52) U.S. Cl. CPC A23J 3/10; A23C 9/15; A23J 3/265; A23J 1/22

(57) ABSTRACT

A fortified high protein food may be produced via an extrusion process whereby acid casein is mixed with whey protein, alkali, water, and one or more additive, which additive may be configured as a vitamin or mineral. Upon exiting the extruder, the mixture may be cut, dried, and/or tempered, milled and screened before being further processed or packaged. The final product may be configured as a crisp high protein food or a powdered high protein food.

### CROSS REFERENCE TO RELATED APPLICATIONS:

This non-provisional utility patent application is a continuation-in-part of and claims priority from U.S. patent application Ser. No. 15/650,737 filed on Jul. 14, 2017, which application is a continuation of and claims priority from U.S. patent application Ser. No. 14/875,463 filed on Oct. 5, 2015 (now U.S. Pat. No. 9,723,859), which application claimed priority from provisional U.S. Pat. App. No. 62/059,355 filed on Oct. 3, 2014. The present patent application also claims priority from provisional U.S. Pat. App. No. 63/200,184 filed on Feb. 19, 2021, all of which applications are incorporated by reference herein in their entireties.

### STATEMENT REGARDING FEDERALLY SPONSORED RESEARCH OR DEVELOPMENT

No federal funds were used to create or develop the invention herein.

### REFERENCE TO SEQUENCE LISTING, A TABLE, OR A COMPUTER PROGRAM LISTING COMPACT DISK APPENDIX

N/A

### BACKGROUND

The health consequences of frequently eating foods that are high in carbohydrates and/or unnatural fats have been widely reported in the last several years. Accordingly, foods that are high in protein have been found to be some of the healthiest options, and many consumers are subsequently seeking out such foods. Where milk proteins are included as part of a higher protein diet, vast improvements have been found with muscle protein synthesis, promoting satiety, preserving and increasing lean muscle mass, enhancing calcium retention and improving bone strength. Additionally, the more convenient a food product is to prepare and eat, the more likely consumers with busy lifestyles are to purchase and/or consume such a food product.

High protein food products that are convenient for consumers to prepare and eat that are currently available to consumers are often based on meat (e.g., beef jerky, poultry jerky, etc.), soy (e.g., roasted edamame, soy “milk”, etc.), or cow's milk (e.g., casein, whey protein). However, prior art high protein foods based on cow's milk often require large amounts of sweeteners (either artificial or natural) so as to be palatable. Additionally, prior art high protein foods based on cow's milk do not provide a liquid cow's milk protein profile at high protein concentrations.

In the context of extruded food products, the addition of milk-derived protein often affects the food product in an undesirable manner. Such effects include but are not limited to contributing to an unpalatable food product, undesirable texture of the food product, and/or an undesirable density of the food product. Accordingly, a need exists for a food product with a high milk protein content that is palatable, and which has a desirable texture and density. In addition, powdered high protein food optimally would be homogeneous and have desirable solubility and heat stability.

Micronutrient malnutrition contributes considerable burden of diseases throughout the world. In 2000, the World Health Report identified iodine, iron, vitamin A and zinc deficiencies as being among the world's most serious health risk factors. In addition to the more obvious clinical manifestations, micronutrient malnutrition is responsible for a wide range of non-specific physiological impairments, leading to reduced resistance to infections, metabolic disorders, and delayed or impaired physical and psychomotor development. The public health implications of micronutrient malnutrition are potentially huge and are especially significant when it comes to designing strategies for the prevention and control of diseases such as HIV/AIDS, malaria and tuberculosis, and diet-related chronic diseases. While micronutrient deficiencies are certainly more frequent and severe among disadvantaged populations, they do represent a public health problem in some industrialized countries. Fortification of food with micronutrients is a valid food-based approach to reduce micronutrient malnutrition when existing food supplies are unable to provide adequate levels of the respective nutrients in the diet.

### BRIEF DESCRIPTION OF THE DRAWINGS

The accompanying drawings, which are incorporated in and constitute a part of this specification, illustrate embodiments and together with the description, serve to explain the principles of the methods and systems.

FIG. 1 is a schematic representation of an illustrative method for making a crisp high protein food according to the present disclosure.

FIG. 2 is a schematic representation of an illustrative method for making a powdered high protein food according to the present disclosure.

FIGS. 3A-3G show various embodiments of a crisp high protein food.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment and/or aspect includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

Before the various aspects of the high protein food are explained in detail, it is to be understood that the present disclosure is not limited to the details of applications, processes, and/or parameters set forth in the following description or illustrated in the drawings unless otherwise indicated in the following claims. The high protein food is capable of other embodiments and of being practiced or of being carried out in various ways. Operational parameters included herein are for illustrative purposes only, and in no way limit the scope of the high protein food unless otherwise indicated in the following claims. The following detailed description is of the best currently contemplated modes of carrying out illustrative embodiments of the invention.

### 1. Illustrative Aspects for One Process for Making a High Protein Food

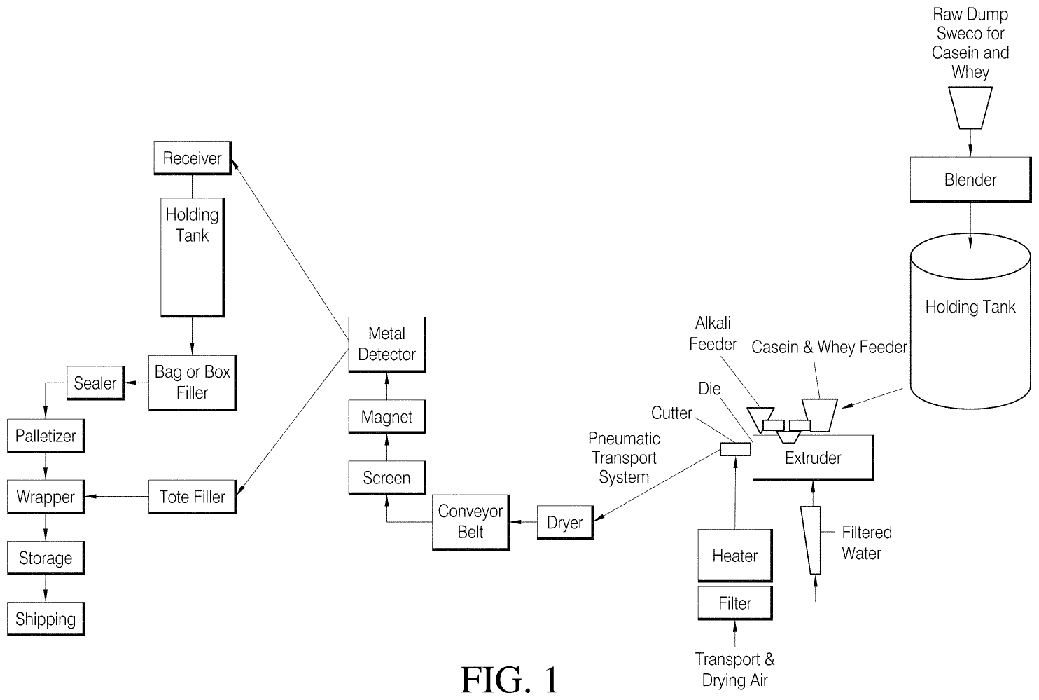

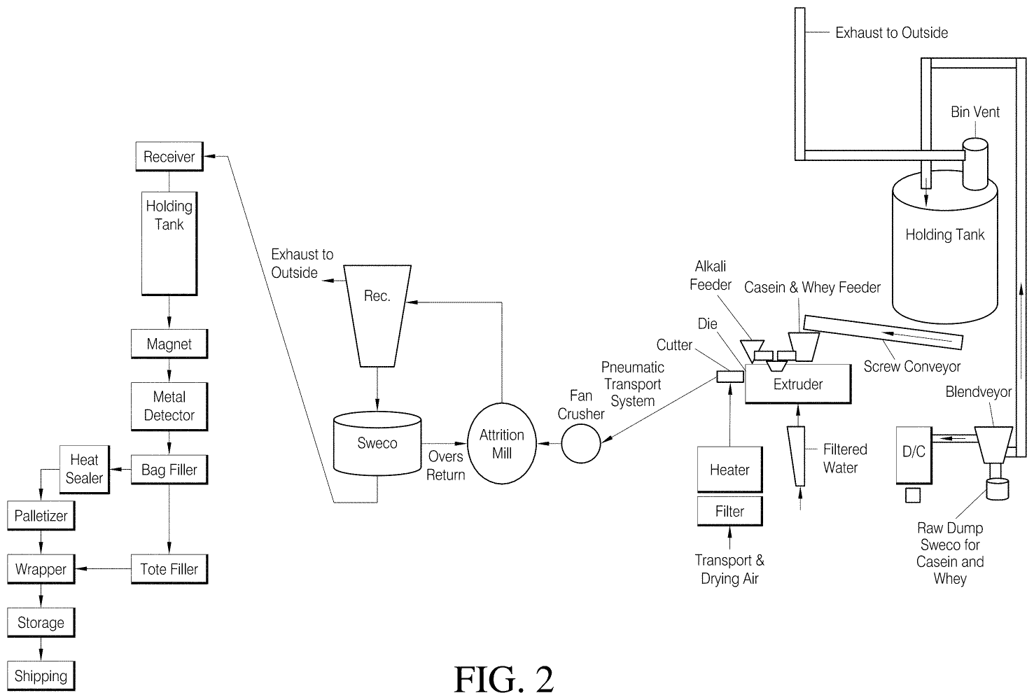

Illustrative aspects for one process for making a high protein food in accordance with the present disclosure is shown schematically in FIG. 1, wherein the high protein food may be configured as a crisp high protein food as described in detail below. Illustrative aspects for another process for making a high protein food in accordance with the present disclosure is shown schematically in FIG. 2, wherein the high protein food may be configured as a powdered high protein food as described in detail below. Although specific reference to operational parameters, equipment, unit operations and the like may be made in the foregoing description, shown in the accompanying drawings, and/or provided in any Appendices included herewith, it is to be understood that those constraints, parameters, and/or values are for illustrative purposes only, and other values and/or parameters may be used when carrying out other aspects of a process for making a high protein food without limiting the scope of the present disclosure unless so indicated in the following claims.

In an aspect of a process for making a high protein food shown in FIG. 1, acid casein may be mixed with whey protein and an alkali. In such a manner, the resulting end product (whether a crisp, fortified, or a powdered high protein food) may be comprised of only whey and casein protein, with no other protein sources therein. That is, the end product (whether a crisp, fortified, or a powdered high protein food) may be free of soy protein, meat-based protein, other plant proteins, etc. Such a protein composition may be necessary to achieve the desired flavor, texture, appearance, scent, and/or other attributes of the end product (whether a crisp, fortified, or a powdered high protein food). Accordingly, in at least one illustrative embodiment, the crisp high protein food, powdered high protein food, and fortified high protein food are configured to contain only whey protein and casein protein, and contain no other types of proteins. Further, the whey and casein proteins may be added such that the ratios thereof approximate the ratios found in cow's milk, as discussed in further detail below. However, in another illustrative embodiments the whey protein may comprise approximately 25% of the total protein content of the end product (whether a crisp, fortified, or a powdered high protein food), with the remainder of the protein comprised of casein.

In another illustrative embodiments the whey protein may comprise approximately 30% of the total protein content of the end product (whether a crisp, fortified, or a powdered high protein food), with the remainder of the protein comprised of casein. In another illustrative embodiments the whey protein may comprise approximately 35% of the total protein content of the end product (whether a crisp, fortified, or a powdered high protein food), with the remainder of the protein comprised of casein. In another illustrative embodiments the whey protein may comprise approximately 40% of the total protein content of the end product (whether a crisp, fortified, or a powdered high protein food), with the remainder of the protein comprised of casein. In another illustrative embodiments the whey protein may comprise approximately 45% of the total protein content of the end product (whether a crisp, fortified, or a powdered high protein food), with the remainder of the protein comprised of casein. In another illustrative embodiments the whey protein may comprise approximately 50% of the total protein content of the end product (whether a crisp, fortified, or a powdered high protein food), with the remainder of the protein comprised of casein. In another illustrative embodiments the whey protein may comprise approximately 55% of the total protein content of the end product (whether a crisp, fortified, or a powdered high protein food), with the remainder of the protein comprised of casein.

The acid casein, whey protein, and alkali may be mixed and/or blended using any suitable method and/or structure, including but not limited to blending tanks, mixers, conveyors, and/or combinations thereof unless otherwise indicated in the following claims. The ingredients may be added individually, separately, and in liquid or dry forms without limitation unless so indicated in the following claims. It is contemplated that for the illustrative aspects, the moisture content of these components may be approximately 6-12% by weight. This mixture may then be introduced into an extruder. Water may also be introduced to the extruder and adjustments made to the alkali, which may cause the pH of the resulting mixture to be approximately neutral. It is contemplated that the water introduced to the extruder may be filtered and/or otherwise purified before it enters the extruder as shown in FIGS. 1 & 2.

It is contemplated that in an aspect of the method, water in the amount of 3-30% by weight may be added to the extruder. If the alkali was not added to the mixture in the previous step, it may be added to the extruder at this point. In an aspect, it is contemplated that the density of the casein and whey protein mixture and the density of the casein, whey protein, and alkali mixture may be from 0.4 to 0.8 g/ml loose and from 0.5 to 1.1 g/ml when packed. However, in another aspect of the present disclosure not pictured herein, wet curd casein or liquid whey protein may be added to the extruder in place of or in addition to solid acid casein or whey protein. In such an aspect, the ratio of solid protein mixed may be lower than if liquid protein is not used. Additionally, in such an aspect, all or a portion of the water necessary for the process may be provided via the liquid whey protein.

In an aspect, it is contemplated that the extruder may be configured as a twin, co-rotating screw extruder, but the extruder may be differently configured in other embodiments and/or aspects without limitation unless so indicated in the following claims. The feed rate of ingredients and water to the extruder will depend at least upon the size of the extruder, and it is contemplated that for some embodiments and/or aspects of a process for making a high protein food the screws speeds may be from 150 RPM to 300 RPM.

Generally, it is contemplated that for the illustrative process, the temperature of the mixture may increase as residence time within the extruder increases. In an aspect, the residence time of the mixture within the extruder may be from about 5 seconds to about 25 seconds, although the scope of the present disclosure is not so limited unless indicated in the following claims. Accordingly, the temperature range in or around the feed zone of the extruder may be approximately 5-60 C, and the temperature range in or around the product zones (or exit zones) may be approximately 40-120 C. However, heat exchangers may be employed to add or remove heat from the extruder (or product therein) at any point such that these values may be different for other aspects of the present process without limitation unless so indicated in the following claims.

As the resulting mixture exits the extruder, the mixture may expand such that the density of the product within the extruder is greater than the density thereof upon exiting the extruder. Additionally, a portion of the water content in the product may flash off due to the pressure differential between the internal portion of the extruder and the exterior of the extruder. If the desired final product is a crisp high protein food (one illustrative example of which is described in detail below), the product may be cut upon exiting the extruder, which may be done via a cutter engaged with the extruder. Additionally, different dies engaged with the extruder may impart different aspects to a crisp high protein food, as described in further detail blow. The cut product pieces may then be tempered and/or dried to achieve the desired final moisture content, which may be approximately 1-14% by weight according to an illustrative aspect. In one illustrative embodiment, the protein content of the end product (whether a crisp, fortified, or a powdered high protein food) may be approximately 90-95% on a dry weight basis. At this protein level the product may be labeled a Milk Protein Isolate (MPI) per the American Dairy Products Institute (ADPI) Concentrated Milk Proteins Standard, which is reproduced below and in Tables 1, 2, & 3.

### ADPI Concentrated Milk Proteins Standard

Product Definition

Concentrated Milk Protein products are obtained by concentrating bovine skim milk through filtration processes so that the finished dry product contains 40% or more protein by weight. Concentrated Milk Protein products may be produced by filtration, dialysis or any other safe and suitable process by which all or part of the lactose and minerals may be removed. Products cannot be produced by combining separately produced casein (caseinate) and whey proteins.

Milk Protein Concentrate (MPC) and Milk Protein Isolate (MPI) are produced by filtration methods (Ultrafiltration and Diafiltration) which capture essentially all the casein and whey proteins contained in the raw material stream in the finished product, resulting in a casein-to-whey protein ratio equivalent to that of the original milk, generally a value of 80:20.

Concentrated Milk Protein products may also be produced using Microfiltration, which will alter the casein-to-whey protein ratio compared to that found in milk. The casein-to-whey protein ratio typically ranges between 82:18 and 95:5 for commercially available products. Where Microfiltration is used, the resulting product is called Microfiltered Milk Protein (MMP) or Micellar Casein (MC).

Composition: MPC and MPI

Several different MPC and MPI products are commercially available, each of which is identified by a number which represents the protein content of the product. These include:

TABLE 1
MPC and MPI compositions per ADPI Concentrated
Milk Proteins Standard.

| Product | Protein % | Fat % | Lactose % | Ash % | Moisture % |
|---|---|---|---|---|---|
| MPC 40 | 39.5 min | 1.25 max | 52.0 max | 10.0 max | 5.0 max |
| MPC 42 | 41.5 min | 1.25 max | 51.0 max | 10.0 max | 5.0 max |
| MPC 56 | 55.5 min | 1.50 max | 36.0 max | 10.0 max | 5.0 max |
| MPC 70 | 69.5 min | 2.50 max | 20.0 max | 10.0 max | 6.0 max |
| MPC 80 | 79.5 min | 2.50 max | 9.0 max | 8.0 max | 6.0 max |
| MPC 85 | 85.0 min* | 2.50 max | 8.0 max | 8.0 max | 6.0 max |
| MPI | 89.5 min* | 2.50 max | 5.0 max | 8.0 max | 6.0 max |

*Protein content ≥85.0% is reported on a dry basis, all other parameters are reported “as is”

Composition: MMP and MC

Several different MMP and/or MC products are commercially available, each of which is identified by a number which represents the protein content of the product. These include:

TABLE 2
MMP and MC compositions per ADPI Concentrated
Milk Proteins Standard.

| Product | | | | | |
|---|---|---|---|---|---|
| MMP/MC | Protein % | Fat % | Lactose % | Ash % | Moisture % |
| 42 | 41.5 min | 1.25 max | 51.0 max | 6.0 max | 5.0 max |
| 70 | 69.5 min | 2.50 max | 16.0 max | 8.0 max | 6.0 max |
| 80 | 79.5 min | 3.00 max | 10.0 max | 8.0 max | 6.0 max |
| 85 | 85.0 min* | 3.00 max | 3.0 max | 8.0 max | 6.0 max |
| 90 | 89.5 min* | 3.00 max | 1.0 max | 8.0 max | 7.0 max |

*Protein content over ≥85.0% is reported on a dry basis, all other parameters are reported “as is”

TABLE 3
Microbiological Standards and Methods of Analysis
per ADPI Concentrated Milk Proteins Standard.

| Parameter | Standard | Test Method |
|---|---|---|
| Standard Plate | 30,000 cfu/g max | FDA BAM |
| Count | | |
| Coliform Bacteria | 10 cfu/g max | AOAC 989.10 (Petrifilm) |
| Salmonella | Neg. | FDA BAM |
| Listeria | Neg. | FDA BAM |
| Yeast/Mold | 100/g max | FDA BAM |
| Moisture | See chart | AOAC 927.05 (Vacuum |
| | | Oven) |
| Milkfat | See chart | AOAC 989.05 (Mojonnier) |
| Protein | See chart | SM 15.132 (Kjeldahl) |
| Casein | See definition | TBD |
| Ash | See chart | AOAC 900.02 (Gravimetric) |
| Lactose | See chart | SM 15.092 (Enzymatic) |

Product Labeling (per ADPI)

Milk Protein Concentrate (MPC) is labeled to reflect the protein content of the finished product. Product labeled as Milk Protein Isolate (MPI) must contain a minimum of 89.5% protein. Microfiltered Milk Protein (MMP) and Micellar Casein (MC) are labeled to reflect their protein content.

Product Applications and Functionality (per ADPI)

MPC, MPI, MMP and/or MC can be used as food ingredients in a variety of food categories. Depending on the food category in which the concentrated milk proteins are used, they can serve as: emulsifiers, flavor enhancers, flavoring agents, formulation aids, humectants, stabilizers and thickeners, texturizers, and sources of high-quality protein.

Storage & Shipping (per ADPI)

Product should be stored and shipped in a cool, dry environment with temperatures below 80° F. and relative humidity below 65%. Stocks should be rotated and utilized within 1-2 years.

Packaging (per ADPI)

Multiwall kraft bags with polyethylene inner liner or other suitable closed container—i.e., “tote bins”, etc.

In another illustrative embodiment, the protein content of the end product (whether a crisp, fortified, or a powdered high protein food) may be at least 85% protein on a dry weight basis. In another illustrative embodiment, the protein content of the end product (whether a crisp, fortified, or a powdered high protein food) may be at least 80% protein on a dry weight basis. In another illustrative embodiment, the protein content of the end product (whether a crisp, fortified, or a powdered high protein food) may be at least 75% protein on a dry weight basis. In another illustrative embodiment, the protein content of the end product (whether a crisp, fortified, or a powdered high protein food) may be at least 70% protein on a dry weight basis. In another illustrative embodiment, the protein content of the end product (whether a crisp, fortified, or a powdered high protein food) may be at least 65% protein on a dry weight basis. In another illustrative embodiment, the protein content of the end product (whether a crisp, fortified, or a powdered high protein food) may be at least 60% protein on a dry weight basis. In another illustrative embodiment, the protein content of the end product (whether a crisp, fortified, or a powdered high protein food) may be at least 55% protein on a dry weight basis. In another illustrative embodiment, the protein content of the end product (whether a crisp, fortified, or a powdered high protein food) may be at least 50% protein on a dry weight basis. In each of the preceding embodiments for a high protein food (whether a crisp, fortified, or a powdered high protein food) it is contemplated that the protein content may be exclusively from casein and whey protein and not contain any other protein source and may have a protein profile similar or identical to that of cow's milk but without limitation unless otherwise indicated in the following claims.

In an aspect, the product may be tempered and/or dried using a fluidized bed dryer or an oven. As shown in FIGS. 1 & 2, both the process for producing the powdered high protein food and the crisp high protein food may include exposing the product to heated air for drying purposes when the product exits the extruder. Additionally, the product may be transported from the extruder via a pneumatic transport system, which system may be configured to remove a portion of moisture from the product during transport. In an aspect of a method for making a crisp high protein food (referring to FIG. 1), the pneumatic transport system may deliver the product to a dryer, which may be any suitable dryer for removing moisture from the crisp high protein food, including but not limited to microwave ovens, convection ovens, fluidized bed dryers, drum driers, forced air dryers, and/or combinations thereof unless otherwise indicated in the following claims.

Still referring to FIG. 1, the product may be transported from a dryer to a screen and product pieces within certain criteria (e.g., length, width, volume, etc.) may be diverted to a primary stream. The product may be exposed to a magnetic field to remove any magnetic materials and/or passed through a metal detector. The product may be placed in a holding tank for packaging and/or further processing, or they may be directly packaged for shipment and/or sale. It is contemplated that a crisp high protein food may be packaged in bags and/or boxes from approximately 25 pounds to approximately 65 pounds, or totes from about 300 pounds to about 800 pounds without limitation unless so indicated in the following claims.

In various aspects, the shape of the crisp high protein food may range from having an oval cross-sectional shape to a disk cross-sectional shape, it may be cylindrical in shape, spherical, it may have an irregular shape that is twisted and/or curved, it may be a combination of pieces, it may include voids or holes, it may be shaped with a pattern and/or may be flat like a conventional chip. In an aspect, the bulk density of a crisp high protein food may be from about 20 g/L to about 400 g/L. However, the scope of the present disclosure is not so limited unless so indicated in the following claims.

Generally, the texture of a crisp high protein food may vary according to several aspects of the method for producing the crisp high protein food. In one aspect, the crisp high protein food may be expanded after extrusion such that the texture thereof is puffy and/or cracker-like. In another aspect, the method may include tempering and/or drying such that the crisp high protein food has a slightly chewy texture. In yet another aspect, the method may include a tempering and/or drying step that results in the crisp high protein food a brittle and/or crunchy texture. Accordingly, the scope of the present disclosure is in no way limited by the specific texture of the crisp high protein food unless so indicated in the following claims.

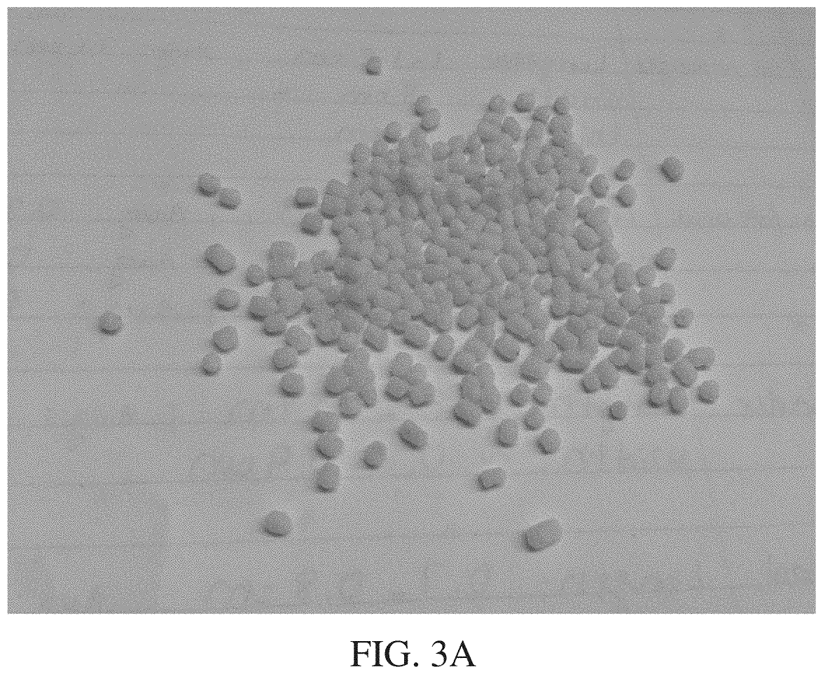

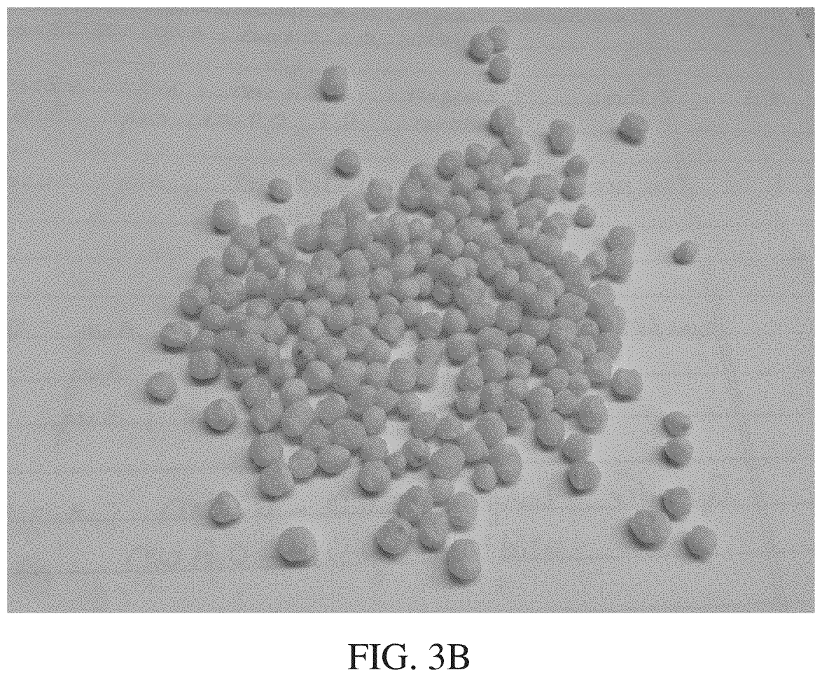

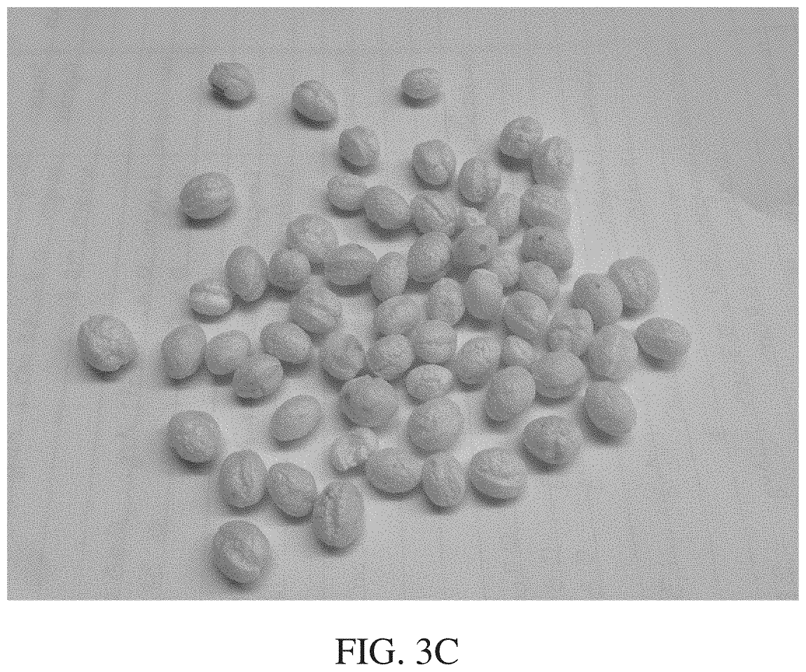

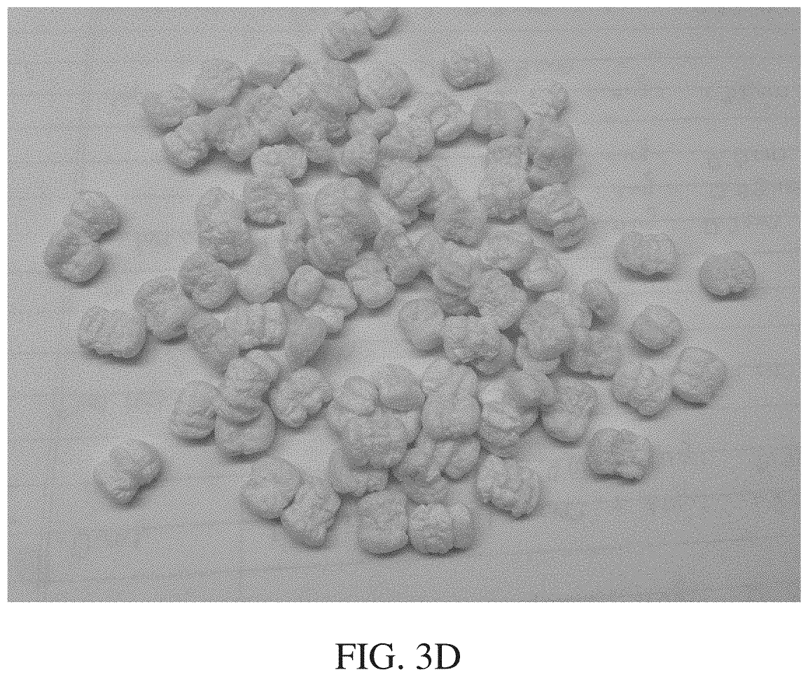

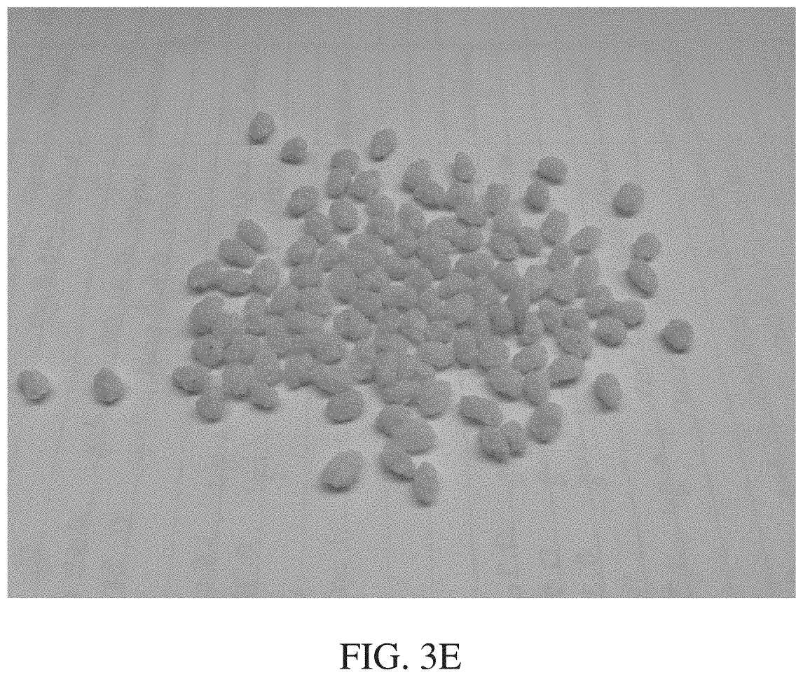

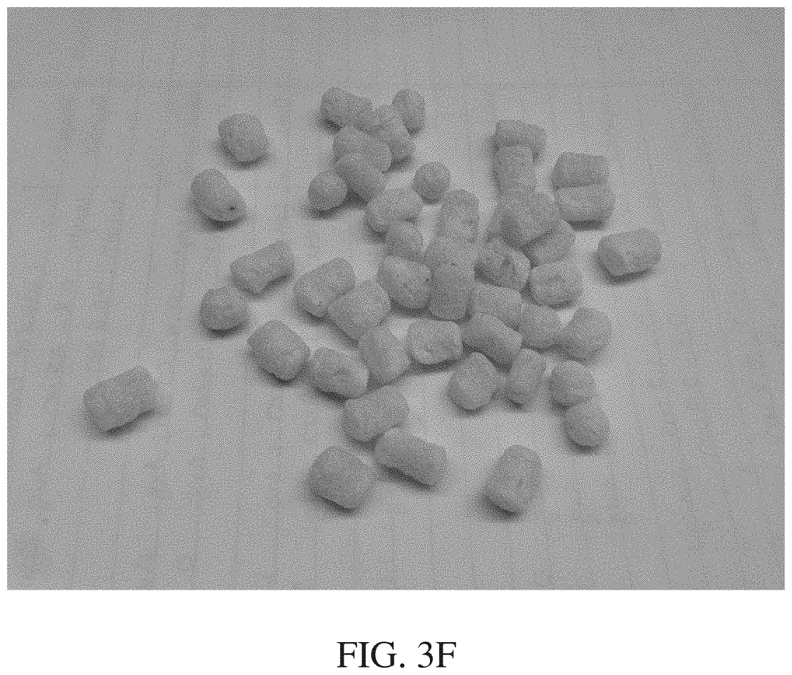

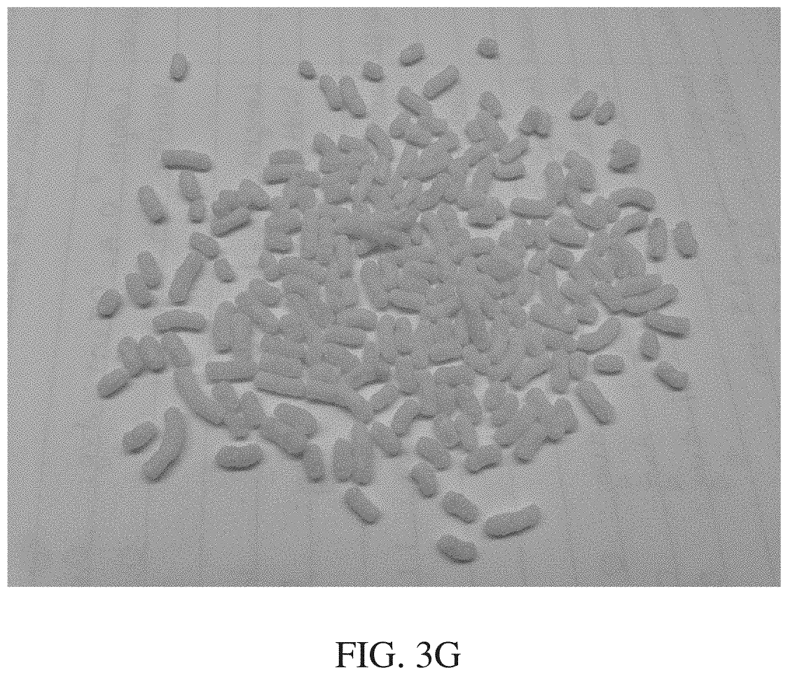

Various illustrative shapes and relative sizes of a crisp high protein food are shown in FIGS. 3A-3G. As shown in FIG. 3A, one aspect of a crisp high protein food may be configured with a very small spherical and/or slightly cylindrical shape, wherein the average diameter thereof may be approximately 0.34 cm. The configuration of a crisp high protein food shown in FIG. 3A may be produced via an extruder with a round die opening of 1.0 mm.

As shown in FIG. 3B, in another aspect of a crisp high protein food, the crisp high protein food may be configured with a small spherical shape, wherein the average diameter thereof may be approximately 0.45 cm. The configuration of a crisp high protein food shown in FIG. 3B may be produced via an extruder with a round die opening of 2.0 mm.

As shown in FIG. 3C, in another aspect the crisp high protein food may be configured with a medium oval shape, wherein the average width may be approximately 0.78 cm and the average length may be approximately 1.20 cm. The configuration of a crisp high protein food shown in FIG. 3C may be produced via an extruder with a slotted die opening having a 1.0 mm width and a 3.0 mm length.

In an aspect of a crisp high protein food shown in FIG. 3D, the crisp high protein food may be configured with a medium combination cylinder shape, wherein the average width may be approximately 0.80 cm and the average length may be approximately 1.1 cm. The configuration of a crisp high protein food shown in FIG. 3D may be produced via an extruder with a slotted die opening having a 1.0 mm width and a 3.0 mm length.

As shown in FIG. 3E, in a further aspect the crisp high protein food may be configured with another medium oval shape, wherein the average width may be approximately 0.53 cm and the average length may be approximately 0.70 cm. The configuration of a crisp high protein food shown in FIG. 3E may be produced via an extruder with a slotted die opening having a 1.0 mm width and a 3.0 mm length.

In another aspect shown in FIG. 3F, the crisp high protein food may be configured with a large cylinder shape, wherein the average width may be approximately 0.85 cm and the average length may be approximately 1.38 cm. The configuration of a crisp high protein food shown in FIG. 3F may be produced via an extruder with a round die opening having a 3.2 mm diameter.

Another aspect of a crisp high protein food is shown in FIG. 3G, wherein the crisp high protein food may be configured with a very small cylinder shape, and wherein the average width may be approximately 0.2 cm and the average length may be approximately 0.82 cm. The configuration of a crisp high protein food shown in FIG. 3G may be produced via an extruder with a round die opening having a 1.0 mm diameter.

Accordingly, the specific shape of the crisp high protein food may depend at least upon the specific die and/or extruder used, and therefore in no way limits the scope of the present disclosure unless so indicated in the following claims. For example, in one aspect the shape of the crisp high protein food may be similar to that of Rice Crispies®, in another aspect thereof the shape may be similar to that of Cheetos®, and in still another aspect thereof the shape may be similar to that of Kix® cereal. Additionally, the scope of the present disclosure is not limited by the specific constraints (such as moisture content, shape, and/or density of the final product), operational parameters, equipment (such as the extruder, die used therewith, etc.), etc. disclosed herein unless so indicated in the following claims. Additional processing parameters for various aspects of a crisp high protein food are shown in Table 4 below.

TABLE 4
Illustrative processing parameters for an illustrative product and method for making same.

| | | Lower Limit | | Target | | Upper Limit | |
|---|---|---|---|---|---|---|---|
| Blending Room Sweco | Screen Size (Mesh) | — | | 1 | inch | — | |
| | Bar Magnet In-Place | — | | Yes | | — | |
| Blendveyor | Blend Time | 80 | seconds | 90 | seconds | 100 | seconds |
| Alkali Dump Station | Screen Size | — | | ½ | inch | — | |
| Extruder | Screw Speed (RPM) | 150 | | 220 | | 300 | |
| | Casein Feed Rate (lbs./hour) | 800 | | — | | 1650 | |
| | Alkali Feed Rate (lbs./hour) | 40 | | — | | 90 | |
| | Water Feed Rate (gallons/hour) | 5 | | — | | 35 | |
| | Product Temperature (C.) | 5 | | 74 | | 120 | |
| Cutter | Number of Knives | — | | 6 | | — | |
| Transport System | Temperature Set point | 50 | | 55 | | 60 | |
| | Actual Temperature | 52 | | 55 | | 61 | |
| Drier | Temperature (C.) | 60 | | 70 | | 80 | |
| | Product Exit Temp. Above | 5 | | 10 | | 15 | |
| | Ambient | | | | | | |
| Screen | Screen Size | ½ | inch | — | | 2 | inch |
| Dust Collector | Air Pressure | — | | 80 | psi | — | |
| | Dust Collector Return to | — | | Yes | | — | |
| | Sweco | | | | | | |
| Holding Tank | | 1,000 | lbs. | 2,000 | lbs. | 25,000 | lbs. |

| | | | | |
|---|---|---|---|---|
| Packing | Magnet in place | — | Yes | — |
| | Metal Detector on and | — | Yes | — |
| | functioning | | | |
| Bag or Box | Weight in lbs. | 30 | — | 55.1 |
| Tote | Weight in lbs. | 400 | — | 700 |

If the desired final product is a powdered high protein food (illustrative aspects of which are described in detail below), the product may be cut, tempered, and then further dried and milled (or ground) to the desired characteristics. In one aspect, cut product may be tempered at a first temperature and then then simultaneously milled (or ground) and dried to the desired moisture content of the final product prior to packaging.

Referring specifically to FIG. 2, the pneumatic transport system previously described may transport the product to a fan crusher, which may be configured to reduce the average particle size of the product. In an aspect, the fan crusher may be configured with a screen size of ¾ of an inch. However, any suitable structure and/or method may be employed to reduce the average particle size of the product, and the scope of the present disclosure is not limited to fan crushers unless so indicated in the following claims. After the fan crusher, the product may be placed in a mill (which may be an attrition mill) to reduce the average particle size of the product. In an aspect, the attrition mill may be configured with an intensity of 57%, a fan set point of 40Hz, and a temperature set point of 65 degrees Celsius. However, any suitable structure and/or method may be employed to reduce the average particle size of the product, and the scope of the present disclosure is not limited to mills and/or mills with the foregoing operational parameters unless so indicated in the following claims.

The product may exit the mill via fluidic pressure, which may transport the product to a solid-gas separator, wherein the gas may be exhausted to the atmosphere and the solid may be placed in a sorting machine (which may be a sifter). In one aspect, the sorting machine may be configured as a Sweco sifter having a screen size of 34T (595 microns). However, the scope of the present disclosure is not so limited unless so indicated in the following claims. The sorting machine may be configured such that a first product stream comprising particle pieces under certain criteria (e.g., length, width, volume, etc.) may be transported to a receiver, and such that a second product stream comprising particle pieces over certain criteria (e.g., length, width, volume, etc.) may be returned to the mill for further average particle size reduction. Although an aspect in the illustrative method shown in FIG. 2 includes two average-particle-size reducing steps (a fan crusher and a mill), the scope of the present disclosure is not so limited, and any number and/or type of average-particle-size reducing methods or apparatuses may be used without limitation unless indicated in the following claims.

Still referring to FIG. 2, the first product stream may be positioned in a holding tank. The first product stream may be exposed to a magnetic field to remove any magnetic materials and/or passed through a metal detector. The product stream may then be packaged for shipment and/or sale. It is contemplated that a powdered high protein food may be packaged in bags and/or boxes from approximately 25 pounds to approximately 65 pounds, or totes from about 300 pounds to about 2204.6 pounds without limitation unless so indicated in the following claims. Additional processing parameters for various aspects of a powdered high protein food are shown in Table 5.

TABLE 5
Illustrative processing parameters for another illustrative product and method for making same.

| | | Lower Limit | | Target | | Upper Limit | |
|---|---|---|---|---|---|---|---|
| Blending Room Sweco | Screen Size (Mesh) | — | | 1 | inch | — | |
| | Bar Magnet In-Place | — | | Yes | | — | |
| Blendveyor | Blend Time | 80 | seconds | 90 | seconds | 100 | seconds |
| Alkali Dump Station | Screen Size | — | | ½ | inch | — | |
| Extruder | Screw Speed (RPM) | 150 | | 220 | | 300 | |
| | Casein Feed Rate (lbs./hour) | 800 | | — | | 1650 | |
| | Alkali Feed Rate (lbs./hour) | 40 | | — | | 90 | |
| | Water Feed Rate (gallons/hour) | 5 | | — | | 35 | |
| | Product Temperature (C.) | 5 | | 74 | | 120 | |
| Cutter | Number of Knives | — | | 6 | | — | |
| Transport System | Temperature Set point | 50 | | 55 | | 60 | |
| | Actual Temperature | 52 | | 55 | | 61 | |
| Fan Crusher | Screen Size | — | | ¾″ | | — | |
| | Intensity (%) | 46 | | 52 | | 59 | |
| Attrition Mill | Intensity (%) | 54 | | 57 | | 60 | |
| | Fan Set Point (Hz) | 40 | | 40 | | 40 | |
| | Temperature Set Point (C.) | 60 | | 65 | | 70 | |
| | Actual Temperature (C.) | 66 | | 69 | | 71 | |
| Sweco Screen | Screen Size | — | | 34T | | — | |
| | Overs to mill | — | | Yes | | — | |
| Dust Collector | Air Pressure | — | | 80 | psi | — | |
| | Dust Collector Return to | — | | Yes | | — | |
| | Sweco | | | | | | |
| Holding Tank | | 4,000 | lbs. | 12,000 | lbs. | 25,000 | lbs. |

| | | | | |
|---|---|---|---|---|
| Packing | Magnet in place | — | Yes | — |
| | Metal Detector on and | — | Yes | — |
| | functioning | | | |
| Bag | Weight in lbs. | 44.1 | — | 55.1 |
| Tote | Weight in lbs. | 700 | — | 1000 |

In one aspect, the final moisture content of the product may be approximately 0.5-12% by weight without limitation, and the milling, grinding, and/or drying process may utilize any suitable structure and/or method to achieve the desired characteristics of the final product unless otherwise limited in the following claims. The specific particle and/or average particle size for any aspect of a powdered high protein food may vary without limiting the scope of the present disclosure unless so indicated in the following claims. However, in one aspect it is contemplated that many applications may require a particle and/or average particle size in the range of 70 to 600 microns. Accordingly, a sifter may be used to achieve the desired particle size and/or average particle size for the final product.

Other ingredients may be extruded with the high protein food. Accordingly, the ingredients that may be introduced to the extruder include, but are not limited to, acid casein, lactic acid casein, rennet casein, sodium caseinate, calcium caseinate, micellar casein, milk protein concentrate (liquid or dried), milk protein isolate (liquid or dried), fresh curd casein (wet), whey protein concentrate (liquid or dried), whey protein isolate (liquid or dried), calcium hydroxide, sodium hydroxide, sodium bicarbonate, potassium bicarbonate, ammonium hydroxide, and sodium citrate without limitation unless otherwise indicated in the following claims.

### 2. Illustrative Aspects a Powdered High Protein Food

Illustrative aspects of a powdered high protein food (which may be produced using the illustrative aspects of a process shown schematically in FIG. 2) will now be described. A typical product data sheet for an illustrative aspect of a powdered high protein food is shown immediately below and in Tables 6 & 7.

Product Description: ECCO Milk Protein Isolate is a highly nutritional product, with a clean flavor and superior functionality. The ECCO Milk Protein Isolate is manufactured continuously to produce a uniform and consistent product with an extremely clean microbiological profile. It is an excellent choice for a variety of food applications.

Physical Characteristics: Uniform, free flowing, granular powder without lumps and foreign particles. White to cream in color, with a bland flavor and odor profile, containing characteristic dairy notes.

TABLE 6
Compositional analysis of an illustrative embodiment of a
high protein food according to the present disclosure.

| Analysis | Specification | Test Method |
|---|---|---|
| Moisture | NMT 6.0% | IR Balance |
| Protein (TN × 6.38)— | NLT 90% | LECO Combustion |
| Dry Basis | | |
| pH (5% Solution) | 6.6-7.3 | GLPR-039 |
| Fat | NMT 2.0 | ISO 5543/IDF 127 |
| Lactose | NMT 3.0 | By difference |

*NMT (not more than) and NLT (not less than)

TABLE 7
Physical analysis of an illustrative embodiment of a high
protein food according to the present disclosure.

| Analysis | Specification | Test Method |
|---|---|---|
| Appearance | Uniform powder | Visual |
| Color | White to cream | Visual |
| Flavor in Solution | Bland with dairy notes | GLPR-026 & GLPR-028 |
| Odor in Solution | Bland with dairy notes | GLPR-026 & GLPR-028 |
| Scorched Particles/ | NMT B Disc/25 g | GLPR-028 |
| Sediment | | |

*NMT (not more than) and NLT (not less than)

A composition analysis, nutritional analysis, mineral analysis, and microbiological analysis for illustrative aspects of a powdered high protein food is provided in Table 8. Also shown in Table 8 are illustrative applications, functionality, packaging, and storage options for various illustrative aspects of a powdered high protein food. As is evident from Tables 6, 7, and 8, illustrative aspects of a powdered high protein food may include a protein profile that is substantially similar to that of typical cow's milk (e.g., the protein may be approximately 80% casein-based and approximately 20% whey-based). However, other ratios of casein-based protein to whey-based protein may be used without limiting the scope of the present disclosure unless so indicated in the following claims.

TYPICAL COMPOSITIONAL ANALYSIS

| | |
|---|---|
| Protein (N × 6.38, Dry basis) % | 93.8 |
| Lactose % | 1.2 |
| Fat % | 1.4 |
| Ash % | 3.3 |
| Moisture % | 4.8 |
| pH (5% Solution) | 7.1 |
| Sediment (25 g) | Disc A |
| Color | White to cream |
| Flavor/Odor | Clean/bland |

TYPICAL NUTRITIONAL ANALYSIS
(g/100 g product)

| | |
|---|---|
| Alanine | 2.9 |
| Arginine | 2.9 |
| Aspartic Acid | 7.0 |
| Cystine | 0.6 |
| Glutamic Acid | 18.9 |
| Glycine | 1.6 |
| Histidine | 2.2 |
| Isoleucine | 4.4 |
| Leucine | 8.0 |
| Lysine | 6.6 |
| Methionine | 2.3 |
| Phenylalanine | 3.9 |
| Proline | 8.9 |
| Serine | 5.0 |
| Threonine | 4.2 |
| Tryptophan | 1.0 |
| Tyrosine | 4.0 |
| Valine | 5.4 |
| Calories | 371 |
| Carbohydrate (Total) | 1.2 |

TYPICAL MINERAL ANALYSIS

| | |
|---|---|
| Calcium % | 0.60 |
| Phosphorus % | 0.70 |
| Potassium % | 0.14 |
| Sodium % | 0.60 |

TYPICAL MICROBIOLOGICAL ANALYSIS

| | |
|---|---|
| Standard Plate Count | <30,000/g |
| Yeast/Mold | <50/g |
| Coliforms | <3 MPN/g |
| E. coli | Not detected/g |
| Salmonella | Not detected/375 g |
| Coag. Pos. Staph | Not detected/g |

Applications include but are not limited to nutritional beverages, frozen desserts, recombined cheeses, yogurt and protein bars.

### Product Functionality

Natural amino acid profile, high solubility, clean flavor profile and heat stability.

### Packaging

Multiwall kraft paper bags with polyethylene inner liner. Net weight 50.0 lbs (22.7 kg) or 44.092 lbs (20 kg).

### Storage

Product should be stored in a cool dry place protected from foreign odors and other contaminants. For extended storage life, store at temperatures below 68° F. (20° C.) with relative humidities below 65%.

Table 8—Illustrative applications, functionality, packaging, and storage options for various illustrative aspects of an illustrative embodiment of a powdered high protein food.

As will be appreciated by a person of ordinary skill in the art, the various values in Appendices C and D for the composition analysis, nutritional analysis, mineral analysis, and microbiological analysis are for illustrative purposes only and are in no way limiting to the scope of the powdered high protein food unless so indicated in the following claims. Those values may be manipulated during the production process, and may be dictated based on the final application of the powdered high protein food. For example, if the end product that includes a powdered high protein food is a nutrition bar, it may be desirable to add protein from other non-milk sources, calcium, other minerals, vitamins, and/or other supplements per daily recommended values and/or market considerations.

In a composition analysis, nutritional analysis, mineral analysis, and microbiological analysis for another illustrative aspect of a powdered high protein food, the mineral composition for the powdered high protein food may be different than that for the powdered high protein food previously described above. For example, Table 9 provides a complete nutritional analysis for various aspects of a powdered high protein food.

TABLE 9
Complete nutritional analysis for an illustrative
embodiment of a high protein food per 100 g.

| | | Source | | | Source |
|---|---|---|---|---|---|
| | | of data | | | of data |
| | A- | C | | A- | C |
| | mount | (calcu- | | mount | (calcu- |
| | per | lated) | | per | lated) |
| | 100 g | A | | 100 g | A |
| | As Is | (analy- | | As Is | (analy- |
| Nutrient | Basis | tical) | Nutrient | Basis | tical) |
| Calories | 367 | C | Phosphorus (mg) | 880 | C |
| Total Fat (g) | 1.4 | A | Iodine (mcg) | | |
| Saturated Fat | 0.98 | C | Magnesium (mg) | 24 | C |
| (g) | | | Zinc (mg) | | |
| Polyunsaturated | 0.04 | C | Selenium (mcg) | | |
| Fat (g) | | | Copper (mg) | | |
| Monoun- | 0.38 | C | Manganese (mg) | | |
| saturated Fat (g) | | | Chromium (mcg) | | |
| Trans-fatty | 0.06 | C | Molybdenum | | |
| acids (g) | | | (mcg) | | |
| Cholesterol | 25 | C | Chloride (mg) | | |
| (mg) | | | Amino Acid | | |
| Sodium (mg) | 908 | C | L-Alanine | 2.83 | A |
| Potassium (mg) | 134 | C | L-Arginine (g) | 3.20 | A |
| Total | 2.4 | C | L-Aspartate (g) | 6.59 | A |
| Carbohydrate | | | L-Cystine (g) | 1.56 | A |
| (g) | | | L-Glutamate (g) | 18.13 | A |
| Dietary Fiber | 0 | C | Glycine (g) | 1.62 | A |
| (g) | | | L-Histidine (g) | 2.32 | A |
| Soluble Fiber | | | L-Isoleucine (g) | 4.52 | A |
| (g) | | | L-Leucine (g) | 8.23 | A |
| Insoluble Fiber | | | L-Lysine (g) | 7.71 | A |
| (g) | | | L-Methionine (g) | 2.39 | A |
| Sugars (g) | 2.4 | C | L-Phenyl- | 4.09 | A |
| Moisture (g) | 3.8 | A | alanine (g) | | |
| Ash (g) | 4.2 | C | L-Proline (g) | 13.08 | A |
| Protein (g) | 88.2 | A | L-Serine (g) | 4.76 | A |
| Vitamin A (IU) | <50 | C | L-Threonine (g) | 4.07 | A |
| Vitamin C (mg) | <1 | C | L-Tryptophan (g) | 1.24 | A |
| Calcium (mg) | 718 | C | L-Tyrosine (g) | 4.42 | A |
| Iron (mg) | 0.64 | C | L-Valine (g) | 4.97 | A |
| Vitamin D (IU) | | | | | |
| Vitamin E (IU) | | | | | |
| Vitamin K | | | | | |
| (mcg) | | | | | |
| Thiamin (mg) | | | | | |
| (g) | | | | | |
| Riboflavin (mg) | | | | | |
| Niacin (mg) | | | | | |
| Vitamin B 6 | | | | | |
| (mg) | | | | | |
| Folic Acid | | | | | |
| (mcg) | | | | | |
| Vitamin B 12 | | | | | |
| (mcg) | | | | | |
| Biotin (mcg) | | | | | |
| Pantothenic | | | | | |
| Acid (mg) | | | | | |

Table 10 provides a complete nutritional analysis for various aspects of another illustrative embodiment of a powdered high protein food.

TABLE 10
Complete nutritional analysis for another illustrative
embodiment of a high protein food per 100 g.

| | | Source | | | Source |
|---|---|---|---|---|---|
| | | of data | | | of data |
| | A- | C | | A- | C |
| | mount | (calcu- | | mount | (calcu- |
| | per | lated) | | Per | lated) |
| | 100 g | A | | 100 g | A |
| | As Is | (analy- | | As Is | (analy- |
| Nutrient | Basis | tical) | Nutrient | Basis | tical) |
| Calories | 367 | C | Phosphorus | 880 | C |
| Total Fat (g) | 1.4 | A | (mg) | | |
| Saturated | 0.98 | C | Iodine (mcg) | | |
| Fat (g) | | | Magnesium | 25 | C |
| Poly- | 0.04 | C | (mg) | | |
| unsaturated | | | Zinc (mg) | | |
| Fat (s) | | | Selenium | | |
| Mono- | 0.38 | C | (mcg) | | |
| unsaturated | | | Copper (mg) | | |
| Fat (g) | | | Manganese | | |
| Trans-fatty | 0.06 | C | (mg) | | |
| acids (g) | | | Chromium | | |
| Cholesterol (mg) | 25 | C | (mcg) | | |
| Sodium (mg) | 43 | C | Molybdenum | | |
| Potassium (mg) | 140 | C | (mcg) | | |
| Total | 2.4 | C | Chloride (mg) | | |
| Carbohydrate (g) | | | Amino Acid | | |
| Dietary Fiber (g) | 0 | C | L-Alanine (g) | 2.83 | A |
| Soluble Fiber (g) | | | L-Arginine (g) | 3.20 | A |
| Insoluble | | | L-Aspartate | 6.59 | A |
| Fiber (g) | | | (g) | | |
| Sugars (g) | 2.4 | C | L-Cystine (g) | 1.56 | A |
| Moisture (g) | 3.8 | A | L-Glutamate | 18.13 | A |
| Ash (g) | 4.2 | C | (g) | | |
| Protein (g) | 88.2 | A | Glycine (g) | 1.62 | A |
| Vitamin A (IU) | <50 | C | L-Histidine (g) | 2.32 | A |
| Vitamin C (mg) | <1 | C | L-Isoleucine | 4.52 | A |
| Calcium (mg) | 1000 | C | (g) | | |
| Iron (mg) | 0.64 | C | L-Leucine (g) | 8.23 | A |
| Vitamin D (IU) | | | L-Lysine (g) | 7.71 | A |
| Vitamin E (IU) | | | L-Meth- | 2.39 | A |
| Vitamin K (mcg) | | | ionme (g) | | |
| Thiamin (mg) | | | L-Phenyl- | 4.09 | A |
| Riboflavin (mg) | | | alamne (g) | | |
| Niacin (mg) | | | L-Proline (g) | 13.08 | A |
| Vitamin B 6 (mg) | | | L-Serine (g) | 4.76 | A |
| Folic Acid (mcg) | | | L-Threonine | 4.07 | A |
| Vitamin B 12 | | | (g) | | |
| (mcg) | | | L-Trypto- | 1.24 | A |
| Biotin (mcg) | | | phan (g) | | |
| Pantothenic | | | L-Tyrosine (g) | 4.42 | A |
| Acid (mg) | | | L-Valine (g) | 4.97 | A |

However, the specific values, ratios, and/or components disclosed herein for any embodiment of a high protein food in no way limit the scope of the present disclosure unless so indicated in the following claims. Accordingly, the specific mineral and/or microbiological content, and/or the specific compositional analysis of the powdered high protein food in no way limits the scope of the present disclosure unless so indicated in the following claims.

As mentioned, illustrative aspects of a powdered high protein food may be produced via the previously described illustrative method for making a high protein food. However, other processes and/or methods may be used without limitation unless otherwise indicated in the following claims. As explained below for a final product crisp high protein food, the density of a final product powdered high protein food may vary from one aspect to the next, which variation may be at least based upon different values for process parameters. However, for the illustrative aspects of a powdered high protein food it is contemplated that the density may be from 0.2 to 0.7 g/ml when loose and from 0.3 to 0.9 g/ml when packed without limitation unless so indicated in the following claims.

### 3. Illustrative Aspects of a Crisp High Protein Food

Illustrative aspects of a crisp high protein food (which may be produced using the illustrative aspects of a process shown schematically in FIG. 1) will now be described. A typical product data sheet for an illustrative aspect of a crisp high protein food is shown in Tables 11-14.

Product Description: ECCO Milk Protein Crisps 90 are highly nutritional crisps, with a clean flavor. This superior protein crisp is manufactured continuously to produce a uniform and consistent product with an extremely clean microbiological profile.

Physical Characteristics: Light and crunchy pieces. White to cream in color, with a bland flavor and odor profile, containing characteristic dairy notes.

TABLE 11
Compositional analysis of an illustrative embodiment of a
high protein food according to the present disclosure.

| Analysis | Specification | Test Method |
|---|---|---|
| Moisture | NMT 6.0% | IR Balance |
| Protein (TN × 6.38)— | NLT 90% | LECO Combustion |
| Dry Basis | | |
| pH (5% Solution) | 6.6-7.3 | GLPR-039 |

*NMT (not more than) and NLT (not less than)

TABLE 12
Physical analysis of an illustrative embodiment of a
high protein food according to
the present disclosure.

| Analysis | Specification | Test Method |
|---|---|---|
| Appearance | Puffed pieces | Visual |
| Color | White to cream | Visual |
| Flavor in Solution | Bland with dairy notes | GLPR-026 & GLPR-028 |
| Odor in Solution | Bland with dairy notes | GLPR-026 & GLPR-028 |
| Scorched Particles/ | NMT B Disc/25 g | GLPR-028 |
| Sediment | | |
| Bulk Density | 150-200 g/L | GLPR-042 |

*NMT (not more than) and NLT (not less than)

TABLE 13
Microbiological analysis of an illustrative embodiment
of a high protein food according to the present disclosure.

| Analysis | Specification | Test Method |
|---|---|---|
| Total Plate Count | NMT 10,000 cfu/g | FDA BAM current edition |
| Yeast | NMT 50 cfu/g | FDA BAM current edition |
| Mold | NMT 50 cfu/g | FDA BAM current edition |
| Coliforms | NMT 10/g | FDA BAM current edition |
| Escherichia coli | Negative/g | FDA BAM current edition |
| Salmonella | Negative/375 g | FDA BAM current edition |

*NMT (not more than) and NLT (not less than)

Ingredient Declaration: Milk Protein Isolate

Packaging: Product is packed in corrugated cardboard with a polyethylene inner liner.

Storage: Recommended to store in a cool, dry, clean environment at temperatures below 68° F. (20° C.) and at a relative humidity below 65%.

Shelf Life: Two years from date of manufacture

Lot Identification: Each lot produced is identified with a six digit production lot code. The first number indicates the production line, the second, third and forth is a Julian date, the fifth is the year and the sixth is the batch number produced on that day.

Country of Origin: USA

Additional Information:
Continuing Guarantee: All processing and packing is performed in a sanitary manner in accordance with all current Good Manufacturing Practice Regulations issued by the Federal Food and Drug Administration and conforms in every respect to the requirements of the Federal Food, Drug and Cosmetic Act as amended and regulations thereof. The product is manufactured to comply with 21 CFR, Sec. 110 (Current Good Manufacturing Practice in Manufacturing, Packing or Holding Human Food).
Allergen Status: The above named product contains dairy products is free from the following allergenic ingredients:
   Cereals containing gluten (e.g. wheat, rye, barley, oats, spelt, karmut or their hybridized strains) and products thereof
   Crustaceans and products thereof
   Eggs and products thereof
   Fish and products thereof
   Peanuts and products thereof
   Soybeans and products thereof
   Tree Nuts (e.g. almonds (Amygdalus communis L.), hazelnuts (Corylus avellana), walnuts (Juglans regia), cashews (Anacardium occidentale), pecan nuts (Carya illinoinensis (Wangenh.) K. Koch), Brazil nuts (Bertholletia excelsa), pistachio nuts (Pistacia vera), macadamia or Queensland nuts (Macadamia ternifolia)) and products thereof
   Celery and products thereof
   Mustard and products thereof
   Sesame seeds and products thereof
   Sulphur dioxide and sulphites at concentrations of more than 10 mg/kg or 10 mg/liter in terms of the total SO2 which are to be calculated for products as proposed ready for consumption or as reconstituted according to the instructions of the manufacturers
   Lupin and products thereof
   Molluscs and products thereof
GMO Status: The product is considered not genetically modified as it is not derived from genetically modified organisms.

Additional Information:
Kosher and Halal Certified
The product is Melamine and Cyanuric Acid free
Manufactured without Genetically Modified ingredients
The product is not manufactured from ingredients derived from animals treated with synthetic bovine somatropin (BST) or recombinant bovine growth hormone (rBGH).
Free from antibiotics
BSE/TSE free
Preservative free and is not with irradiation, fumigation or ozone treatment
In accordance with the United States' 7CFR205.105(g), the materials, which comprise the above-named product, have not produced and handled with the use of sewage sludge

Table 14—Additional information that may be provided on a product data sheet for an illustrative embodiment of a high protein food according to the present disclosure.

A typical composition analysis, nutritional analysis, mineral analysis, and microbiological analysis for illustrative aspects of a crisp high protein food is provided in Table 15. Also shown in Table 15 are illustrative applications, functionality, packaging, and storage options for the illustrative aspects of a crisp high protein food.

TYPICAL COMPOSITIONAL ANALYSIS

| | |
|---|---|
| Protein (N × 6.38, Dry basis) % | 93.8 |
| Lactose % | 1.2 |
| Fat % | 1.4 |
| Ash % | 3.3 |
| Moisture % | 4.8 |
| pH (5% Solution) | 7.1 |
| Sediment (25 g) | Disc A |
| Color | White to cream |
| Flavor/Odor | Clean/bland |

TYPICAL NUTRITIONAL
ANALYSIS (g/100 g product)

| | |
|---|---|
| Alanine | 2.9 |
| Arginine | 2.9 |
| Aspartic Acid | 7.0 |
| Cystine | 0.6 |
| Glutamic Acid | 18.9 |
| Glycine | 1.6 |
| Histidine | 2.2 |
| Isoleucine | 4.4 |
| Leucine | 8.0 |
| Lysine | 6.6 |
| Methionine | 2.3 |
| Phenylalanine | 3.9 |
| Proline | 8.9 |
| Serine | 5.0 |
| Threonine | 4.2 |
| Tryptophan | 1.0 |
| Tyrosine | 4.0 |
| Valine | 5.4 |
| Calories | 371 |
| Carbohydrate (Total) | 1.2 |

TYPICAL MINERAL ANALYSIS

| | |
|---|---|
| Calcium % | 1.10 |
| Phosphorus % | 0.09 |
| Potassium % | 0.15 |
| Sodium % | 0.72 |

TYPICAL MICROBIOLOGICAL ANALYSIS

| | |
|---|---|
| Standard Plate Count | <30,000/g |
| Yeast/Mold | <50/g |
| Coliforms | <3 MPN/g |
| E.coli | Not detected/g |
| Salmonella | Not detected/375 g |
| Coag. Pos. Staph | Not detected/g |

### Applications

Including but not limited to protein bars, cereals and snack products.

### Product Functionality

Natural amino acid profile, high solubility, clean flavor profile, and heat stability.

### Packaging

Corrugated cardboard with a polyethylene liner. Net weight 22.046 lbs (10 kg) to 661.38 lbs (300 kg).

### Storage

Product should be stored in a cool dry place protected from foreign odors and other contaminants. For extended storage life, store at temperatures below 68° F. (20° C.) with relative humidities below 65%.

Table 15—Illustrative applications, functionality, packaging, and storage options for various illustrative aspects of an illustrative embodiment of a crisp high protein food.

As is evident from Table 15, the illustrative aspects of a crisp high protein food may include a protein profile that is substantially similar to that of typical cow's milk (e.g., the protein may be approximately 80% casein-based and approximately 20% whey-based). However, other ratios of casein-based protein to whey-based protein may be used without limiting the scope of the present disclosure unless so indicated in the following claims.

As will be appreciated by a person of ordinary skill in the art, the various values in Table 15 for the composition analysis, nutritional analysis, mineral analysis, and microbiological analysis are for illustrative purposes only and are in no way limiting to the scope of the crisp high protein food unless so indicated in the following claims. Those values may be manipulated during the production process, and may be dictated based on the final application of the crisp high protein food. For example, if the end product that includes a crisp high protein food is a snack product or nutrition bar, it may be desirable to add other ingredients, including but not limited to minerals, vitamins, and/or other supplements per daily recommended values and/or market considerations unless otherwise indicated in the following claims. Accordingly, the specific mineral and/or microbiological content, and/or the specific compositional analysis of the crisp high protein food in no way limits the scope of the present disclosure unless so indicated in the following claims.

As mentioned, the illustrative aspects of a crisp high protein food may be produced via the previously described illustrative aspects of one method for making a high protein food. However, other processes and/or methods may be used without limitation unless so indicated in the following claims. Applicant has found that the density of a final product crisp high protein food may vary depending on various factors, including but not limited to the size and shape of individual morsels as well as the water content added during the extrusion process and the moisture content of the final product unless otherwise indicated in the following claims. It is contemplated that many aspects of the crisp high protein food may have an average density of 0.01-0.50 g/ml.

### 4. Illustrative Embodiments of a Fortified High Protein Food

In one illustrative embodiment, a fortified high protein food may be produced using a process very similar to the illustrative processes shown schematically in FIGS. 1 & 2. Generally, the illustrative embodiments of a fortified high protein food relates to a method for producing a high protein food with an additive, which may produce a mineral/vitamin fortified high protein food (depending on what specific additive is employed) by mixing proteins with at least one additive (e.g., minerals, vitamins and/or other compounds and/or other components such as acids, alkalis, water, etc.) without limitation unless otherwise indicated in the following claims.

Fortification of dried milk and flavored milk powders may be achieved through adding vitamins (A, D, E) and/or minerals (calcium, iron, zinc). However, some processes used in the prior art to concentrate macronutrients may begin to deplete micronutrients, which depletion may occur during the preparation of various high-protein concentrates and isolates such as but not limited to milk protein isolate, pea protein isolate, and soy protein isolate. While containing high levels of protein, fortification of a crisp high protein food as disclosed herein with minerals, vitamins, and/or other nutraceuticals may benefit the consumer. The process for producing a fortified high protein food may be configured such that the concentration of macronutrients does not deplete micronutrients.

The optimal level at which nutrients will be added to a high protein food may depend on a number of factors, including but not limited to levels of consumption and nutritional requirements of the target population; the effect of added nutrients on the functional or sensory (odor, flavor, color, etc.) characteristics of the crisp high protein food; and/or the stability of the nutrients during processing and storage of the fortified high protein food without limitation unless otherwise indicated in the following claims. The disclosure relating to a fortified high protein food may apply to either a crisp high protein food or a powdered high protein food as previously described in detail above without limitation unless otherwise indicated in the following claims. Generally, in one illustrative method the simplest way to fortify a crisp high protein food may be to blend dry forms of vitamins and minerals with the proteins prior to processing, although oily forms can also be added. Unlike liquids, dried proteins can be fortified either prior to or after the heat treatment.

The following is focused on the function of potential minerals and vitamins (which vitamins or minerals may be added to a crisp or powdered high protein food in accordance with the present disclosure to create a fortified high protein food) on the human body and bioavailable compounds. Generally, a fortified high protein food produced according to the present disclosure may be configured with any amount and any number of the proceeding vitamins and/or minerals, or any other additive that may be beneficial for a specific application without limitation unless otherwise indicated in the following claims and according to various process and/or chemical constraints as noted herein or later discovered. Additionally, the specific chemical formula for the additive, mineral, and/or vitamin (e.g., salt, solid, liquid, variant, etc.) in no way limits the scope of the present disclosure unless otherwise indicated in the following claims, and the optimal form thereof may vary from one application to the next. Any specific weight percentages, volume percentages, recommended daily allowances, recipes, etc. provided herein are for illustrative purposes only and are not meant to be limiting to the scope of the fortified high protein food disclosed herein unless otherwise indicated in the following claims.

Iron: Iron is primarily involved in the transfer of oxygen from the lungs to tissues. However, iron also plays a role in metabolism as a component of some proteins and enzymes. Seventy percent of iron in the human body is found in red blood cell hemoglobin and muscle cell myoglobin. Iron deficiency is a common cause of too few healthy red blood cells in the body (anemia). In a pregnant woman, iron deficiency puts the baby at risk of developmental delays. it has been found that diets of infants were as much as 50% deficient in iron, and those of young girls and women up to 54 years of age as much as 30% to 35% below recommended allowances. Regarding fortification, iron is the most challenging nutrient to use for fortification. Generally soluble iron compound has maximum bioavailability, but it may cause rancidity and discoloration. Food sources of iron are less well utilized than inorganic compounds. Regarding biological availability, insoluble ferric orthophosphate has 14% biological value of ferrous sulphate iron added at 20 mg/kg. Thus, use of more insoluble iron is more acceptable for fortification purpose. However, insoluble ferric orthophosphate may become more bioavailable during storage of the products. Water soluble iron compounds (ferrous sulphate, ferrous gluconate, ferrous lactate, etc.) may also be a preferred choice for fortification in certain applications due to highly solubility in gastric juices without limitation unless otherwise indicated in the following claims.

Magnesium: Magnesium helps to activate vitamin D, which in turn regulates calcium and phosphate homeostasis to influence the growth and maintenance of bones. People may suffer from vascular calcification with inadequate magnesium levels in the body. Optimum magnesium levels can work as replacement of low level of Vitamin D and can reduce the chance of osteoporosis. While RDA for magnesium is 420 mg for male and 320 mg for female, a standard diet in the United States contains only half of that amount. As a result, Vitamin D remains unused in the absence of magnesium in 50% of Americans. The average use of magnesium from a diet by the human body is 40-50%, and its absorption increases in the presence of B6 vitamin, lactose, and protein. Magnesium aspartate, magnesium chloride, magnesium Citrate, and magnesium lactate are bioavailable forms of magnesium that can be used to fortify crisp high protein foods as disclosed herein.

Calcium: Calcium plays many important roles in the human body such as formation of bone and teeth, muscle contraction, normal functioning of many enzymes, blood clotting, and normal heart rhythm. To maintain a normal level of calcium in the blood, an adult, pregnant and lactating woman needs to consume minimum 1300 mg calcium/day, whereas children from 1 to 3 years of age need 700 mg/day, and infants need 260 mg/day. Dairy products are an excellent source of dietary calcium, which can be further fortified with calcium salts to achieve an even higher calcium intake per serving. Increased awareness of higher calcium intake has enhanced the business of dairy products enriched with additional calcium content.

Bioavailable forms recommended for the fortification of infant formulas and complementary foods include the carbonate (it can liberate CO₂ in acid systems), the chloride, the citrate and the citrate malate, the gluconate, the glycerophosphate, the lactate, the mono-, di- and tribasic phosphates, the orthophosphate, the hydroxide, and the oxide. All of these salts are either white or colorless. The calcium content of commercially available salts ranges from 9% (the gluconate) to 71% (the oxide). In general, absorption of added calcium is similar to that naturally present in foods, which ranges from about 10% to 30%. However, high levels of calcium inhibit the absorption of iron from foods and so this too is something that needs to be taken into consideration when deciding how much calcium to add. The co-addition of ascorbic acid can help overcome the inhibitory effect of calcium on iron absorption.

Zinc: Zinc, which plays an important role in the maintenance of normal bones and is sometimes referred to as the ‘new calcium’ in fortified dairy beverages. Along with bone health benefits, zinc also contributes to normal brain function, fertility, and DNA synthesis. The positive impact of zinc supplementation on the growth of some stunted children, and on the prevalence of selected childhood diseases such as diarrhea, suggests that zinc deficiency is likely to be a significant public health problem, especially in developing countries. The bioavailability of zinc is dependent at least partially on dietary composition, and in particular on the proportion of high-phytate foods in the diet. At relatively high ratios (e.g., above 15:1), zinc absorption from food may be low, that is to say, less than 15%. Thus, reducing phytic acid content in food may increase bioavailability of zinc. On the other hand, relatively high levels of dietary calcium (e.g., >1 g per day), which might be consumed by some individuals, can inhibit zinc absorption, especially in the presence of phytates. Fortification of zinc is limited to infant formula milk, complimentary foods, and breakfast cereals. In Turkey, fortified bread with zinc was able to increase the growth rate of school children with low-plasma zinc. In multiple forms of zinc (e.g., sulfate, chloride, gluconate, oxide, and the stearate) may be suitable for food fortification without limitation unless otherwise indicated in the following claims. Tri-zinc citrate is also bioavailable form of zinc that can be used to fortify a crisp high protein food according to the present disclosure.

Vitamin B₁₂: Vitamin B₁₂ is vital in the synthesis of amino acid methionine, which is necessary for cell metabolism and survival. Deficiency of this vitamin can lead to defective immune function, impaired neurological deterioration, megaloblastic anemia, and elevated plasma homocysteine. It can cause severe developmental delays in children and young kids. The United States and Canada have recommended that elderly people should consume Vitamin B12 intake as fortified foods or supplements.

Biotin: Biotin is an essential component of enzymes that affects metabolizing fats and carbohydrates, influencing cell growth, and affecting amino acids involved in protein synthesis. Biotin also assists in various metabolic reactions involving the transfer of carbon dioxide and maintaining a steady blood sugar level. Biotin is often recommended as a dietary supplement for strengthening hair and nails.

Vitamin D: Vitamin D is perhaps the most important regulator that controls calcium and phosphate homeostasis to influence the growth and maintenance of bones. It also plays an important role in cell differentiation and in the secretion and metabolism of hormones, including parathyroid hormone and insulin. Vitamin D can be synthesized at most animals' skin including humans under the influence of sunlight. Vitamin D deficiency is fairly common worldwide. Severe deficiency causes rickets in children and osteomalacia in adults. Dietary requirements for vitamin D are increased because the ability of the skin to synthesize this vitamin decreases with age as well as for darker-skinned people whose skin is less capable of synthesizing vitamin D when exposed to sunlight. Vitamin D fortified milk and margarine are well accepted products among people.

Vitamin C: Vitamin C, comprised of ascorbic acid and dehydroascorbic acid, is an important antioxidant. It helps in maintaining collagen formation. Vitamin C also increases the absorption rate of non-haem iron from foods. Severe deficiency of Vitamin C can cause Scurvy which is very rare now. However, prevalence of low or marginal deficiency is still high. Germinated grain, fresh fruit, vegetables and offal are good sources of Vitamin C. However, Vitamin C is very unstable when exposed to high heat, metal, humidity, or an alkaline environment. Therefore, Vitamin C can be depleting fast during cooking. Populations with less access to fresh fruits and vegetables depended on cooked food and infants who are fed on cow's milk are more prone to Vitamin C deficiency. In Chile, it is mandatory to add Vitamin C in food fortified with iron. Recommended daily allowance of Vitamin C is 90 mg for adults. As Vitamin C is very unstable at high temperatures, cold food can be a good carrier of this vitamin. Encapsulated Vitamin C is also an option for fortifying already heat-treated food.

Vitamin E: Vitamin E is a fat-soluble antioxidant which helps to prevent the chronic diseases from free radicals. It also boosts immunity function by inhibiting the activity of protein kinase C which is involved in cell proliferation and differentiation in smooth muscle cells, platelets, and monocytes. Vitamin E inhibits the platelet aggregation. Recommended daily allowance of Vitamin E is 15 mg for adults. Nuts, seeds and vegetable oil are good sources of Vitamin E. Severe Vitamin E deficiency may cause poor transmission of nerve impulses, muscle weakness, and retinal degeneration that leads to blindness. Margarine, fat spreads, and breakfast cereal are some foods that are commonly fortified with Vitamin E.

Folic Acid/Folate (Vitamin B₉): Folic Acid/Folate (Vitamin B₉) plays a central role in cell multiplication and tissue growth via synthesis and methylation of nucleotides. Low intake of folic acid increase the risk of megaloblastic anemia, cardiovascular diseases, cancer and impaired cognitive function in adults. Intake of folic acid is mandatory during pregnancy as deficiency of folic acid can be associated with a higher risk of giving birth to infants with neural tube defects. Populations that have a high intake of refined cereals (which are low in folate) and a low intake of leafy greens and fruits (which are high in folate) are more prone to folic acid deficiency. As a result, addition of folic acid to enriched grain products in the United States was mandated in 1998. The required fortification level is 154 μg/100 g flour.

Other B Vitamins: Other Vitamin B group compounds include Thiamin (Vitamin B₁, Riboflavin (Vitamin B₂), Niacin (Vitamin B₃), and pyridoxine (Vitamin B₆). Deficiency in one B vitamin can lead to deficiencies in other B vitamins too. Refining of cereal grains removes almost all the thiamine (Vitamin B₁), riboflavin (Vitamin B₂) and niacin (Vitamin B₃). Thus, fortification of foods with these particular nutrients is needed to eliminate vitamin B deficiencies and their associated diseases (i.e. beriberi and pellagra). Thiamine (Vitamin B₁) is a cofactor for several key enzymes involved in carbohydrate metabolism and is also directly involved in neural function. Severe deficiency in Thiamine can cause beriberi. Riboflavin (Vitamin B₂) is a precursor of various nucleotides which act as coenzymes in various metabolic pathways and in energy production. Niacin (nicotinic acid or Vitamin B₃) works as a functional group of the coenzymes which is essential for oxidative processes and its deficiency results in pellagra. Vitamin B₆ works as a carbonyl-reactive coenzyme to various enzymes which are involved in the metabolism of amino acids. Riboflavin and Vitamin B₆ deficiencies are frequently associated with deficiencies in one or more of the other B-complex vitamins.

### Micronutrient Stability

Vitamins may be sensitive to heat, light, and humidity, as well as oxidizing and reducing agents to different degrees. Minerals (e.g., magnesium, calcium, iron, zinc, iodine, phosphorous, selenium, copper, manganese, chloride, chromium, molybdenum, etc.) are, in general, are less sensitive than vitamins (e.g., Vitamin A, Vitamin D, Vitamin E, Vitamin K, Thiamin, Riboflavin, Niacin, Vitamin B6, Folic Acid, Vitamin B12, Biotin, Pantothenic Acid, etc.) to physical and chemical factors. Nevertheless, they are reactive in nature and must be selected after considering possible interactions with milk proteins, potential adverse effects on the sensory properties of milk, and the bioavailability of the mineral form. Most vitamins and minerals show retention of 70 to 100% after a single common industrial heat treatment. However, repeated heat treatments can result in extensive losses. Vitamin C, which is easily degraded by oxygen and light, is the exception. To compensate for these losses, it may be advantageous to include add an appropriate overage of each micronutrient during fortification. One illustrative example of recommended overages based on processing losses alone for common types of milk are presented in Table 16. Losses during storage may vary at least with time, temperature, humidity, and exposure to light, and should be determined locally and the optimal amount of overages may vary from one application to the next, and are therefore in no way limiting to the scope of the present disclosure unless otherwise indicated in the following claims.

TABLE 16
Recommended overages (%) for selected nutrients
for dried milk based on losses during processing.

| Nutrient | Overages (IU/L) |
|---|---|
| Vitamin A | 40 |
| Vitamin D | 40 |
| Vitamin E | 20 |
| Vitamin B₁ | 20 |
| Vitamin B₂ | 20 |
| Niacin | 20 |
| Vitamin B₆ | 20-30 |
| Vitamin B₁₂ | 40 |
| Folate | 40 |
| Vitamin C | 50 |
| Iron | 5 |
| Calcium | 5 |

Applicant has found through testing and experimentation that it was possible to nutritionally fortify high protein foods produced using the method schematically shown in FIGS. 1 & 2 without degrading the quality and functional characteristics of the high protein food while inhibiting degradation of the additive (e.g., vitamin, mineral, etc.) while processing and maintaining bioavailability in the final product. It has been found that in one application it may be especially advantageous to configure the fortified high protein food as a fortified crisp high protein food without limitation unless otherwise indicated in the following claims. An illustrative side-by-side comparison of an unfortified crisp high protein food (column labeled “Protein Crisp”) and two illustrative embodiments of nutritionally fortified crisp high protein foods (columns labeled “Protein Crisp+” and “Protein Crisp ++” respectively) is provided in Table 17. Again, the percentages, amounts, specific additives, etc. for the fortified crisp high protein food shown in Table 17 are for illustrative purposes only, and in no way meant to be limiting to either the fortified high protein food or method for producing same unless otherwise indicated in the following claims. From experimentation it has been found that an illustrative embodiment of a method for producing a fortified high protein food may result in retention of at least 77% percent of an additive and in some cases may result in 100% retention without limitation unless otherwise indicated in the following claims.

TABLE 17
Illustrative nutritional profile for three illustrative embodiments of a fortified high protein food.

| | | Protein Crisp | | Protein Crisp+ | | Protein Crisp++ | |
|---|---|---|---|---|---|---|---|
| | | | % | | % | | % |
| Nutrient | RDI* | per 30 g | RDI | per 30 g | RDI | per 30 g | RDI |
| Energy (kcal) | | 115 | | 115 | | 115 | |
| Protein (g) | 56 | 26 | 47 | 26 | 47 | 26 | 47 |
| Total Carbohydrate (g) | 130 | 1 | 1 | 1 | 1 | 1 | 1 |
| Sugar | — | 1 | — | 1 | — | 1 | — |
| Dietary Fiber | 38 | 0 | 0 | 0 | 0 | 0 | 0 |
| Soluble Fiber | — | 0 | — | 0 | — | 0 | — |
| Insoluble Fiber | — | 0 | — | 0 | — | 0 | — |
| Fat (g) | — | 1 | — | 1 | — | 1 | — |
| Saturated | — | 1 | — | 1 | — | 1 | — |
| Polyunsaturate Fat | — | 0 | — | 0 | — | 0 | — |
| Monounsaturated Fat | — | 0 | — | 0 | — | 0 | — |
| Trans-Fatty Acid | — | 0 | — | 0 | — | 0 | — |
| Cholesterol (mg) | — | 11 | — | 11 | — | 11 | — |
| Moisture (gm) | — | 1.1 | — | 1.1 | — | 1.1 | — |
| Ash (gm) | — | 1 | — | 1 | — | 1 | — |
| Sodium (mg) | 1500 | 45 | 3 | 50 | 3 | 50 | 3 |
| Potassium (mg) | 4700 | 42 | 1 | 1410 | 30 | 2820 | 60 |
| Magnesium (mg) | 420 | 6 | 1 | 126 | 30 | 252 | 60 |
| Iron (mg) | 8 | 0 | 2 | 2 | 30 | 5 | 60 |
| Zinc (mg) | 11 | 0 | 0 | 3 | 30 | 7 | 60 |
| Calcium (mg) | 1000 | 300 | 30 | 300 | 30 | 600 | 60 |
| Iodine (mcg) | 150 | 0 | 0 | 45 | 30 | 90 | 60 |
| Phosphorus (mg) | 700 | 207 | 30 | 210 | 30 | 420 | 60 |
| Solenium (mcg) | 55 | 0 | 0 | 17 | 30 | 33 | 60 |
| Copper (mcg) | 900 | 0 | 0 | 270 | 30 | 540 | 60 |
| Manganese (mg) | 2.3 | 0 | 0 | 1 | 30 | 1 | 60 |
| Chloride (mg) | 2300 | 0 | 0 | 690 | 30 | 1380 | 60 |
| Chromium (mcg) | 35 | 0 | 0 | 11 | 30 | 21 | 60 |
| Molybdenam (mcg) | 45 | 0 | 0 | 14 | 30 | 27 | 60 |
| Vitamin A (mcg) | 900 | 9 | 1 | 270 | 30 | 540 | 60 |
| Vitamin D (mcg) | 15 | 0 | 2 | 5 | 30 | 9 | 60 |
| Vitamin C (mg) | 90 | 0 | 0 | 27 | 30 | 54 | 60 |
| Vitamin E (mg) | 15 | 0 | 0 | 5 | 30 | 9 | 60 |
| Vitamin K (mcg) | 120 | 0 | 0 | 36 | 30 | 72 | 60 |
| Thiamin (mg) | 1.2 | 0.0 | 0 | 0.4 | 30 | 0.7 | 60 |
| Riboflavin (mg) | 1.3 | 0.0 | 0 | 0.4 | 30 | 0.8 | 60 |
| Niacin (mg) | 16 | 0 | 0 | 5 | 30 | 10 | 60 |
| Vitamin B6 (mg) | 1.3 | 0.0 | 0 | 0.4 | 30 | 0.8 | 60 |
| Folic Acid (mg) | 0.4 | 0.0 | 0 | 0.1 | 30 | 0.2 | 60 |
| Vitamin B12 (mcg) | 2.4 | 0.0 | 0 | 0.7 | 30 | 1.4 | 60 |
| Biotin (mcg) | 30 | 0 | 0 | 9 | 30 | 18 | 60 |
| Pantothenic Acid (mg) | 5 | 0 | 0 | 2 | 30 | 3 | 60 |

*Dietary Reference Intakes (DRIs): Recommended Dietary Allowances and Adequate Intakes, Elements and Dietary Reference Intakes (DRIs): Recommended Dietary Allowances and Adequate Intakes, Vitamins for males aged 31 to 50 years.

In another illustrative embodiment of a fortified high protein food (either crisp or powder), the fortifying component may be introduced to the high protein food after extrusion rather than during the extrusion process as described above. In a first illustrative embodiment of post-extrusion addition of one or more fortifying components, the fortifying component(s) may be added as part of a flavoring system. The flavoring system may be configured such that an oil and/or slurry of oil that includes one or more flavoring agents and one or more fortifying agents is applied to a dried high protein food (typically a crisp but without limitation unless otherwise indicated in the following claims). The application of the oil and/or slurry may be accomplished via a spraying mechanism within a rotating drum, but the scope of the present disclosure is not so limited unless otherwise indicated in the following claims.

In an embodiment wherein an oil is applied to a dried high protein food, first the oil may be applied to the high protein food in a rotating drum. After the oil has sufficiently coated the high protein food, one or more dry flavoring agents and/or one or more fortifying components may be added to the rotating drum such that the drying flavoring agent(s) and/or fortifying component(s) adhere to the surface of the high protein food due to the presence of oil thereon. it is contemplated that a post-extrusion addition of a fortifying agent may allow for better incorporation, less degradation, and/or higher bioavailability of heat-sensitive fortifying components.

In addition to fortifying components and flavoring agents, masking agents may be incorporated into any of the high protein foods disclosed herein or produced by a process disclosed herein. It is contemplated that such masking agents may be configured to compensate for any flavor impact to the end product, which flavor impact may be caused by a fortifying component and/or production technique without limitation unless otherwise indicated in the following claims.

### 5. Illustrative Embodiments of a Textural Manipulation for High Protein Foods

Manipulating various alkalis and alkali combinations along with the potential addition of carbon dioxide and/or dry ice may allow production of various textured high protein foods/crisps using extrusion technology. Controlling and altering the alkali combinations with or without carbon dioxide/dry ice may allow for the development of softer or firmer, denser or lighter, glassier or foamier, and/or more or less porous textured extruded crisp high protein foods, which can be useful when producing various types of protein foods/snacks. Generally, the optimal alkali and/or alkali combination will vary from one embodiment of a high protein food to the next, and may be dependent at least upon the final product for which the high protein food is used. Accordingly, any suitable alkali and/or alkali combination, including but not limited to sodium bicarbonate, sodium carbonate, sodium sequicarbonate, sodium hydroxide, other sodium-based compounds, ammonium hydroxide, calcium hydroxide, calcium carbonate, other calcium-based compounds, magnesium carbonate, potassium hydroxide, potassium carbonate, other potassium-based compounds and/or combinations thereof may be used with the high protein food without limitation unless otherwise indicated in the following claims.

While this alkali manipulation alone can be useful to developing a softer crisp high protein food, this approach can also help offset other steps including fortification of crisp high protein foods, which could potentially create harder crisp high protein foods. Typical means of softening extruded crisp high protein foods rely on the addition of a non-protein component such as starch, which dilutes the protein itself in the crisp high protein food and can increase the carbohydrate portion of the food/snack. Carbon dioxide and/or dry ice addition may protect heat-liable fortifying agents, such as Vitamin C (as described above), in addition to nucleating and generating pores within the extruded crisp high protein food and/or resulting food product. In one illustrative, non-limiting example, decreasing added calcium hydroxide from approximately 1.6% to 0% while at the same time increasing sodium bicarbonate from roughly 0.3% to 1% decreased bulk density 50 g/L in curl-shaped crisp high protein food applications. Such a decrease in bulk density is often in line with softer texture and more aerated structure. Other methods and/or components may be used to achieve the desired texture without limitation unless otherwise indicated in the following claims.

In another illustrative example, adding dry ice at 1 to 5% by weight decreased the bulk density of a crisp high protein food by about 10 g/L. Dry ice incorporation increased occluded air and radial expansion index (REI). For example, REI was 3.0 at 0% dry ice and increased to 3.2 when 5% by weight dry ice was added. However, in other embodiments the REI may be 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4,9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0% or even as high at 10%, 15%, or 20% without limitation unless otherwise indicated in the following claims. These changes, which resulted from carbon dioxide yet could be accomplished by any other inert gas, may soften the high protein crisp texture and may reduce glassiness when compared to a control sample. In experiments, the dry ice was added as chips, but any suitable form factor for an inert gas and/or phase (solid, liquid, gas) may be used without limitation unless otherwise indicated in the following claims. Additionally, the dry ice or other inert gas material may be added at any suitable step in the manufacturing process of the crisp high protein food without limitation unless otherwise indicated in the following claims, though in one illustrative embodiment it may be especially advantageous to add the dry ice to the extruder with the whey and casein protein.

It is contemplated that the sublimation and/or evaporation of the gas may cause increased cavities in certain areas of the crisp high protein food, and/or cavities of differing sizes, positions, and/or concentrations. Accordingly, the scope of the present disclosure is not limited to carbon dioxide at the ratios specifically mentioned above, and/or the specific effects thereof, and other amounts of carbon dioxide and/or other inert compounds may be used to manipulate the texture of the resulting high protein food without limitation unless otherwise indicated in the following claims.

Generally, it is contemplated that the texture a crisp high protein food (both fortified and unfortified) may be a function of process and machine variables. For example, through experimentation, Applicant has found that the texture of a crisp high protein food may become less glassy and more foam-like with smaller pores and may become softer as the portion of sodium bicarbonate, as a percentage of the total alkali blend, increases and the level of calcium hydroxide addition decreases. Applicant has also found that generally more expansion may occur (especially with a crisp high protein food configured as a loop) as casein particle size becomes finer. Various extruder characteristics, such as screw speed and configuration, barrel and product temperature, die throughput per open area, and others may also have an effect on overall texture. For example, underworking the material at the die end of the extruder may lessen expansion, increase bulk density, and firm up texture.

Although the descriptions of the illustrative aspects of the present disclosure have been quite specific, it is contemplated that various modifications could be made without deviating from the spirit and scope of the present disclosure. Accordingly, the scope of the present disclosure is not limited by the description of the illustrative aspects and/or corresponding figures unless so indicated in the following claims.

In the foregoing detailed description, various features are grouped together in a single embodiment for purposes of streamlining the disclosure. This method of disclosure is not to be interpreted as reflecting an intention that the present disclosure requires more features than are expressly recited in each claim. Rather, as the following claims reflect, inventive aspects lie in less than all features of a single foregoing disclosed embodiment. Thus, the following claims are hereby incorporated into this detailed description, with each claim standing on its own as a separate embodiment.

The materials used to construct the apparatuses and/or components thereof for a specific process will vary depending on the specific application thereof, but it is contemplated that polymers, synthetic materials, metals, metal alloys, natural materials, and/or combinations thereof may be especially useful in some applications. Accordingly, the above-referenced elements may be constructed of any material known to those skilled in the art or later developed, which material is appropriate for the specific application of the present disclosure without departing from the spirit and scope of the present disclosure unless so indicated in the following claims.

Having described preferred aspects of the various processes and apparatuses, other features of the present disclosure will undoubtedly occur to those versed in the art, as will numerous modifications and alterations in the embodiments and/or aspects as illustrated herein, all of which may be achieved without departing from the spirit and scope of the present disclosure. Accordingly, the methods and embodiments pictured and described herein are for illustrative purposes only, and the scope of the present disclosure extends to all processes, apparatuses, and/or structures for providing the various benefits and/or features of the present disclosure unless so indicated in the following claims.

While the high protein foods, processes for making high protein foods, components thereof, and apparatuses therefor have been described in connection with preferred aspects and specific examples, it is not intended that the scope be limited to the particular embodiments and/or aspects set forth, as the embodiments and/or aspects herein are intended in all respects to be illustrative rather than restrictive. Accordingly, the processes and embodiments pictured and described herein are no way limiting to the scope of the present disclosure unless so stated in the following claims.

Although several figures are drawn to accurate scale, any dimensions provided herein are for illustrative purposes only and in no way limit the scope of the present disclosure unless so indicated in the following claims. It should be noted that the welding processes, apparatuses and/or equipment therefor, and/or welded substrates produced thereby are not limited to the specific embodiments pictured and described herein, but rather the scope of the inventive features according to the present disclosure is defined by the claims herein. Modifications and alterations from the described embodiments will occur to those skilled in the art without departure from the spirit and scope of the present disclosure.

Any of the various features, components, functionalities, advantages, aspects, configurations, process steps, process parameters, etc. of a high protein food or process for making same may be used alone or in combination with one another depending on the compatibility of the features, components, functionalities, advantages, aspects, configurations, process steps, process parameters, etc. Accordingly, a nearly infinite number of variations of the present disclosure exist. Modifications and/or substitutions of one feature, component, functionality, aspect, configuration, process step, process parameter, etc. for another in no way limit the scope of the present disclosure unless so indicated in the following claims.

It is understood that the present disclosure extends to all alternative combinations of one or more of the individual features mentioned, evident from the text and/or drawings, and/or inherently disclosed. All of these different combinations constitute various alternative aspects of the present disclosure and/or components thereof. The embodiments described herein explain the best modes known for practicing the apparatuses, methods, and/or components disclosed herein and will enable others skilled in the art to utilize the same. The claims are to be construed to include alternative embodiments to the extent permitted by the prior art.

Unless otherwise expressly stated in the claims, it is in no way intended that any process or method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including but not limited to: matters of logic with respect to arrangement of steps or operational flow; plain meaning derived from grammatical organization or punctuation; the number or type of embodiments described in the specification.

What is claimed is:

1. A fortified high protein food produced by a process of extruding a mixture of acid casein, whey protein, water, and an alkali capable of neutralizing said acid casein, wherein said alkali is selected from a group consisting of sodium bicarbonate, sodium carbonate, sodium sequicarbonate, sodium hydroxide, ammonium hydroxide, calcium hydroxide, calcium carbonate, magnesium carbonate, potassium hydroxide, and potassium carbonate, wherein said fortified high protein food contains at least 60 to 65% protein by weight on a dry basis, wherein a protein content of said fortified high protein food is from 15% to 35% by weight whey protein and from 65% to 85% by weight casein, wherein said fortified high protein food has a protein profile similar to that of cow's milk, wherein said fortified high protein food does not include a protein source other than said casein and said whey protein, and wherein said fortified high protein food is fortified with a first nutrient.

2. The fortified high protein food according to claim 1 wherein said first nutrient is selected from a group consisting of a mineral and a vitamin.

3. The fortified high protein food according to claim 2 wherein said mineral is selected from a group consisting of magnesium, calcium, iron, zinc, iodine, phosphorous, selenium, copper, manganese, chloride, chromium, and molybdenum.

4. The fortified high protein food according to claim 3 wherein said vitamin is selected from a group consisting of Vitamin A, Vitamin D, Vitamin E, Vitamin K, Thiamin, Riboflavin, Niacin, Vitamin B6, Folic Acid, Vitamin B12, Biotin, and Pantothenic Acid.

5. The fortified high protein food according to claim 1 wherein said high protein food is further defined as having a moisture content from about 1% to about 14% by weight.

6. The fortified high protein food according to claim 1 wherein said high protein food is further defined as having a bulk density from about 20 g/L to about 400 g/L.

7. A method for producing a fortified high protein food, said method comprising the steps of:
a) creating a mixture of from 65 to 85 percent by weight of a casein protein and from 15 to 35 percent by weight of a whey protein;
b) introducing said mixture into an extruder;
c) adding water to said extruder to create a second mixture such that said second mixture is from 3 to 30 percent by weight water;
d) adding a nutrient to said extruder to create a third mixture;
e) forcing said third mixture through a die engaged with said extruder to create a plurality of cut pieces;
f) allowing said third mixture to expand in an environment with ambient pressure and temperature; and,
g) removing moisture from said plurality of cut pieces such that the moisture content of said plurality of cut pieces is from 0.5% (one-half percent) to 12% (twelve percent) by weight, wherein a protein content of said plurality of cut pieces is 60% (sixty percent) or greater by weight, and wherein said fortified high protein food produced by said method does not include a protein source other than said casein protein and said whey protein.

8. The method according to claim 7 wherein said mixture has a moisture content from 3% to 13% by weight.

9. The method according to claim 8 wherein said second mixture has a moisture content less than 30% by weight.

10. The method according to claim 9 wherein said extruder is further defined as being a twin, co-rotating screw extruder.

11. The method according to claim 10 further comprising the step of removing heat energy from said second mixture.

12. The method according to claim 11 wherein said protein content is further defined as comprising from 15% to 35% by weight whey protein and from 65% to 85% by weight casein.

13. The method according to claim 7 wherein said method is further defined such that a residence time of said third mixture within said extruder is less than 10 seconds.

14. The method according to claim 7 wherein said fortified high protein food is further defined as having a bulk density from 20 g/L to 400 g/L.

15. The method according to claim 7 wherein a temperature of said third mixture within said extruder does not exceed 75 C.

16. The method according to claim 15 further comprising the step of adding dry ice to said second mixture prior to said step of forcing said third mixture through said die.

17. A method for adjusting a texture of a high protein food, said method comprising the steps of:
a) creating a mixture of from 65 to 85 percent by weight of a casein protein and from 15 to 35 percent by weight of a whey protein;
b) introducing said mixture into an extruder;
c) adding water to said extruder to create a second mixture such that said second mixture is from 3 to 30 percent by weight water;
d) adding dry ice to said extruder to create a third mixture;
e) forcing said third mixture through a die engaged with said extruder to create a plurality of cut pieces;
f) allowing said third mixture to expand in an environment with ambient pressure and temperature; and,
g) removing moisture from said plurality of cut pieces such that the moisture content of said plurality of cut pieces is from 0.5% (one-half percent) to 12% (twelve percent) by weight, wherein a protein content of said plurality of cut pieces is 90% (ninety percent) or greater by weight, and wherein said high protein food produced by said method does not include a protein source other than said casein protein and said whey protein.

18. The method according to claim 17 wherein said crisp high protein food is further defined as having a radial expansion index of at least 3.2% but less than 20%.

19. The method according to claim 18 wherein said second mixture has a moisture content less than 30% by weight.

20. The method according to claim 19 wherein said fortified high protein food is further defined as having a bulk density from 20 g/L to 400 g/L.
